(19) United States
(12) Patent Application Publication
(10) Pub. No.: US 2011/0104137 A1
(43) Pub. Date: May 5, 2011

(54) ADJUNCTS AND COMPLEXES FOR IMPROVING HMG-CoA REDUCTASE INHIBITOR (STATIN) AND SELECTIVE PHOSPHODIESTERASE 5 INHIBITOR THERAPY

(75) Inventors: Kenneth T. Richardson, Anchorage, AK (US); Don C. Pearson, Lakewood, WA (US)

(73) Assignee: ChronoRX LLC, an Alaska Limited Liability Company, Anchorage, AK (US)

(21) Appl. No.: 12/762,639

(22) Filed: Apr. 19, 2010

Related U.S. Application Data
(60) Provisional application No. 61/171,313, filed on Apr. 21, 2009.

(51) Int. Cl. A61K 31/122; C07C 279/14; C07D 475/04; C07C 49/753; C07D 339/04; C07C 215/40; A61K 31/385; A61K 31/519; A61K 31/205; A61K 31/40; A61K 31/496; A61P 15/10; A61P 3/00

(52) U.S. Cl. 424/94.1; 562/560; 544/258; 568/377; 549/39; 562/567; 514/440; 514/249; 514/556; 514/423; 514/252.16

(57) ABSTRACT

Dosage forms and methods of use are disclosed for a) adjuncts administered individually or simultaneously with HMg-CoA reductase inhibitors (statins) and/or selective phosphodiesterase 5 inhibitors or, b) the administration of a conjugate consisting of the adjuncts and an HMG-CoA reductase inhibitor and/or a selective phosphodiesterase 5 inhibitor. The invention is useful in the amelioration of side effects associated with HMG-CoA reductase inhibitors and will improve their effectiveness in diseases for which these are useful. The invention will also improve the effectiveness in the of selective phosphodiesterase 5 inhibitors in patients using these medications alone or in conjunction with statins, for the treatment of erectile dysfunction.

### CROSS-REFERENCE TO RELATED APPLICATION

This application claims the benefit of U.S. Provisional Patent Application No. 61/171,313, filed Apr. 21, 2009, the contents of which are incorporated herein by reference.

### BACKGROUND OF THE INVENTION

1. Field of the Invention

This invention is in the field of pharmacology and relates to dosage formulations, which contain adjunctive ingredients for use in the pharmacological treatment of patients currently using an HMG-CoA reductase inhibitor (statin), or that may be useful for patients requiring such treatment, and methods of treatment for patients using phosphodiesterase 5 inhibitors with or without the simultaneous use of statins. It describes therapeutic uses of individual or complexed physiological molecules that favorably modify cell physiology in order to augment statin therapy (or reduce undesirable statin side effects) and improve the efficiency of the phosphodiesterase 5 inhibitors. The formulations described by the invention may include a statin and/or a phosphodiesterase inhibitor (PDE5 inhibitor) with the adjuncts.

### THE INVENTION INCLUDES . . .

| STATINS | ADJUNCT COMPONENTS |
|---|---|
| Lovastatin | Tetrahydrobiopterin |
| Pravastatin | L-Arginine |
| Simvastatin | Acetyl-L-Carnitine |
| Fluvastatin | Coenzyme Q10 |
| Atorvastatin | D-a-Lipoic acid |
| Rosuvastatin | PDE5 inhibitors |
| Pitavastatin | |

The last major revision (2001) of the US guidelines for the use of statins increased the number of Americans for whom statins are recommended from 13 million to 36 million, most of whom do not yet have significant heart disease but are estimated to be at moderately elevated risk of developing coronary heart disease(1). Many others are taking HMG-CoA reductase inhibitors in essence as prophylaxis for a variety of non-cardiac diagnoses. The use of this class of drugs is expected to continue to increase at a rapid pace. It is appropriate to define formulations to increase the effectiveness of these medications and to address their undesirable side effects—this patent describes appropriate formulations that do both.

This document is organized in the following manner
A review of cholesterol biochemistry and metabolism.
A review of statin biochemistry and metabolic activities.
Individual discussions of the adjunct components, their biochemistry and biological interactions.
A discussion of PDE5 inhibitors and the relationship of these to the statin family.

2. Description of the Prior Art and the Present Invention

Isoprene is a five-carbon hydrocarbon (C5H8), which in the plant and animal kingdom is used as the basis for the formation of isoprenoids, a large and varied group of hydrocarbon molecules that include many medically relevant compounds, e.g., steroids (the most common of which is cholesterol), fat-soluble vitamins and coenzymes (such as Coenzyme Q10) that are synthesized within the cell . . . and the prenylated¹ proteins. ¹Polymerization of isoprene molecules; often providing a basis for attachment to proteins, facilitating their attachment to receptors.

In animals these isoprenoids are biosynthesized via the mevalonate (MVA) pathway (See detailed description infra). Metabolic inhibitors of the MVA pathway, HMG-CoA reductase inhibitors (statins) are widely used for the treatment of hypercholesterolemia.

Humans require cholesterol (CHOL). It is present in and integral to the production, structure and function of all cell membranes in the body, including those of the mitochondria. It is essential for the synthesis of steroid hormones (cortisone, estrogen, testosterone, etc.), for steroid-related molecules such as vitamin D, and for the production of bile acids.

The liver processes about 1,000 mg of CHOL daily, of which 800 mg become bile salts that are secreted (along with bicarbonate) into the duodenum to alkalinize chyme—the latter is partly digested food that is passed from the stomach into the duodenum—from pH 2 to pH 6.5, which is optimal for fat digestion. Of equal importance, bile salts (See infra) further aid in fat digestion by enhancing fat solubilization via the production of an emulsion of microscopic micelles, which are essential for the intestinal absorption of fat-soluble vitamins and of CHOL. These micelles consist predominantly of triglycerides and cholesterol esters coated by phospholipids. The latter permit ester migration across the lipid-rich microvillus membrane lining of the small intestine, ultimately into the hepatic portal vein whence they are carried to the liver.

Triglycerides and CHOL are then assembled intracellularly in enterocytes as chylomicrons stabilized by a shell of phospholipid, CHOL and protein. The major protein involved in this activity is apolipoprotein B-48 (a truncated form of apolipoprotein B-100), which is essential for the assembly, secretion and subsequent metabolism of chylomicrons.(2) The size of chylomicrons is inversely related to biliary secretion(3); bile salts increase the efficiency of intestinal absorption of CHOL by reducing micelle size.

Of the total 1,000 mg of CHOL daily processed by the liver about 200 mg is incorporated into liver-manufactured lipoproteins for its transport in the blood to tissues throughout the body.

CHOL is more concentrated in tissues in which it is highly synthesized or in those tissues that have more densely-packed membranes (e.g., cells with a large number of mitochondria), such as liver, spinal cord and brain. All brain CHOL is derived from de novo synthesis.(4;5) This organ, however, does ultimately interact with the liver since the brain's “used” CHOL is converted to 24-S-hydroxycholesterol (hydrophilic, MW 402), which traverses the blood-brain barrier and is transported to the liver by plasma lipoproteins (probably high density lipoproteins) for final disposal.

The Liver

Although the liver processes about 1 gram of CHOL each day less than 20% comes from diet: the remainder is synthesized in the liver.(6) When dietary intake of CHOL increases the liver makes less. Conversely when dietary sources are decreased a healthy liver makes more CHOL.

Human life can't exist without CHOL, but too much in the wrong form, critically located, creates health problems. For example: When CHOL is directly oxidized in the phospholipid bilayer of the vascular endothelium or when its oxidized ester is accumulated by macrophages that in turn embed in the vascular endothelium, atherosclerosis is the result. Low density lipoprotein (LDL) (7) consists of several fragments(8); LDL apolipoprotein B-100(9-12), CHOL, cholesteryl esters(13;14) and the fatty acids occurring in the cell from cholesteryl ester hydrolysis. All of these can be oxidized. Such oxidation creates yet more free radicals that damage cells (especially cell membranes). This damage often stimulates secondary inflammation, which in turn compounds the destructive oxidative process.

Furthermore the plasma membrane of cells contains both polyunsaturated and monounsaturated lipids, both of which are susceptible to oxidative damage by free-radical processes.(15;16)

While it is impossible to know the relative pathologic consequence of each of these free radical sources in a given situation it is evident that their composite importance is great.

Metabolism of Cholesterol

CHOL is a lipid produced in all eukaryotic cells. It resides in the plasma and mitochondrial cell membranes of all tissues. It is an amphipathic sterol, resulting from the conjugation of a hydrophobic steroid and a hydrophilic alcohol. Although amphipathic, its solubility in water is limited and cannot be readily transported in the plasma. Therefore it requires liposomal packaging in the liver for transport purposes.

CHOL plays a central role in human biochemistry (17) since it is necessary for multiple physiological functions (fluidity, permeability, synaptic transmission, etc.) that involve all cell membranes—including not only plasma membranes but also the outer and inner mitochondrial membranes. CHOL is not only universally critical for the synthesis and maintenance of cellular membranes it is also involved with and necessary for trans-membrane cell signaling. This it performs via the formation of lipid rafts in the plasma membrane combined with invaginated caveolae and clathrin-coated pits. The latter are required for the caveolae/clathrin-dependent trans-membrane endocytic activities that are utilized by a most cells.

Cellular internalization of lipoproteins by means of endocytosis is dependent not only upon an energy-dependent process via the clathrin receptors, but also upon passive transfer via physical gaps in the endothelium (about 26 nm).

Lipoprotein particle sizes vary:
chylomicrons 7-24 nm;
HDL 8-10 nm;
LDL 20-27 nm;
VLDL particles 40-50 nm(3;18).

The larger the particle in each group the less efficient the trans-membrane transit.

As addressed elsewhere: Important statin events occur not just in the liver but can be of equal or greater consequence in all other cells of the body—each with a continuously change in requirement, each influence by its own dynamic, local microenvironment.

In re CHOL, the liver is a manufacturing and packaging plant that sends LDL laden with cholesteryl esters (and triglycerides) into the general circulation, so that each cell abutting the circulatory path has available a supply of plasma LDL cholesteryl ester, appropriate to the cell type and its individual state of maturity. And, most importantly, the supply is available at that given instant in time when the cell is not endogenously producing enough cholesterol to meet its immediate physiological needs.

The liver is designed to transport LDL cholesteryl esters in packages that are big enough so they cannot insinuate (and be sequestrated) between vascular endothelial cells. They are therefore available, upon request from each cell, for the delivery of these esters across the plasma membrane of the cell via a clathrin-containing, energy-requiring endosomal transport system and receptors that each cell manufactures upon demand.

A presumption is that the “requesting” cells have the appropriate enzyme system (in this case, cholesterol ester hydrolase) to convert cholesteryl esters of otherwise lower value, perhaps extracted from endosomal LDL, into the more useful cholesterol the cell requires.

Unfortunately cholesteryl esters love to be oxidized under the sheets of vascular endothelial cells with damaging atherosclerotic consequences. Once oxidized and contained in small LDL packages they can slip between vascular endothelial cells or be carried in by macrophages.

It is this complex world of CHOL transport and manipulation that the HMG-CoA reductase inhibitors (statins) are designed to influence and that the invention is designed to impact.

Bile Acids

Bile acids (bile salts) are polar derivatives of CHOL. They are still amphipathic but are more polar, more water-soluble than CHOL itself.

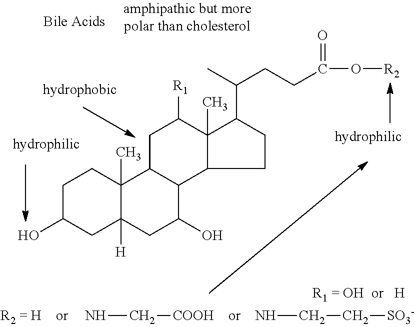

The liver disposes of CHOL by two routes: excretion into the intestine and by secretion into the plasma for delivery to tissues.

The intestinal excretion and absorption/reabsorption of CHOL is dependent upon the bile—a complex fluid containing water, electrolytes, bile acids, CHOL, phospholipids and bilirubin—produced in the liver and released into the duodenum. Bile acids emulsify fat globules into smaller micelles, increasing the surface area accessible to lipid-hydrolyzing enzymes. The only mechanism by which CHOL can be either excreted into or absorbed by the intestine is by way of hepatic bile secretions containing these bile salts and CHOL. Eventually both are reabsorbed by the small intestine, returned to the liver via the portal vein, and usually re-excreted—the enterohepatic cycle. Agents that interrupt this cycle can modify CHOL levels. These agents include: 1) synthetic resins as well as soluble fiber that bind bile acids and/or CHOL and prevent absorption/reabsorption; 2) drugs that act on epithelial cells lining the lumen of the small intestine and inhibit absorption of CHOL.

While dietary CHOL may be obtained from intestinal absorption most CHOL is synthesized de novo in hepatocytes. The most important synthesis for the purposes of this invention is the conversion of Acetyl-Coenzyme A into 3-hydroxy-3-ethylglutarylcoenzyme A (HMG-CoA). The latter is subsequently converted to a CHOL precursor, mevalonic acid, by the enzyme HMG-CoA reductase in the endoplasmic reticulum membrane (19). This is the first and rate-limiting enzyme in a multi-step biosynthetic pathway that leads to CHOL. (See diagram infra).

There are other closely associated and shared important intermediate metabolic pathways for the synthesis of various isoprenoids (e.g., the coenzymes Q) (20) that are recognized and taken advantage of by the invention.

Statins and Cholesterol Synthesis

Statins lower circulating CHOL levels by inhibiting HMG-CoA reductase; thus reducing CHOL synthesis in all cells, including hepatocytes. This overall reduction of cholesterol synthesis results in lower plasma CHOL levels—in part because the liver produces less for transport into the blood stream—but perhaps in larger part because all cells of the body can extract cholesterol from the blood stream to meet their individual needs when their own cholesterol production is inhibited by a statin.

Importantly, reduced intracellular cholesterol upregulates expression of the LDL receptor gene, increasing LDL receptor expression and receptor-mediated endocytosis of LDL into individual cells—thus lowering LDL in the blood stream.(21;22).

HMG-CoA synthase activity is mostly located in the inner membrane of mitochondria.(23) Since the number of mitochondria varies significantly in different cell types, it is understandable that at least in this regard the cellular effect of HMG-CoA reductase inhibitors can be variable from cell to cell.

Acyl CoA: Cholesterol Acyl Transferase (ACAT)

Acyl-CoA cholesteryl acyl transferase (ACAT) in the endoplasmic reticulum(24) transfers acyl groups from one molecule to another; in this case converting CHOL and fatty acids into CHOL esters (cholesteryl ester). ACAT is a critical enzyme for bile acid production and for the CHOL esterification required in the liver for plasma transport to tissues throughout the body.

Within many cells free CHOL is converted into cholesteryl esters for storage and movement inside the cell. Cholesteryl esters can then be hydrolyzed in cytosol-based endosomes/lysosomes by the enzyme cholesteryl ester hydrolase (CEH) to release CHOL. A balance between ACAT, CEH and HMG-CoA reductase maintains intracellular CHOL homeostasis(20). Hepatocytes and cells elsewhere can switch this molecular form instantly if they have ACAT (CHOL→cholesteryl ester) and CEH to cleave the CHOL ester (cholesteryl ester→CHOL).

ACAT specifically plays an important role in foam cell formation and atherosclerosis by participating in the production and accumulation of cholesteryl esters in macrophages embedded in the vascular intima.

Dyslipidemias

Dyslipidemias are defined as:
Total cholesterol, LDL-cholesterol, triglyceride, apoB-100, or Lp(a) level above the ninetieth percentile; or
HDL-cholesterol level below the tenth percentile for the general population.

Apolipoprotein B-100—(apoB-100)

Apolipoprotein B-100 (apoB-100) is the major protein in LDL and contains a ligand for binding LDL to a cell surface receptor.(26) Each native LDL particle contains a single apoB-100 molecule in an elongated form that wraps partly around the LDL particle that, along with the polar ends of lipoprotein phospholipids and surface CHOL, permits aqueous circulation in plasma.

Because the apoB-100 attached to an LDL particle develops immunoreactivity when the LDL phospholipids are oxidized, it is a useful immunochemical marker for monitoring the extent of LDL oxidation.(12). Oxidized phospholipids are invariably present within atherosclerotic plaques. It is likely that the immunoreactivity of apoB-100 is a causative factor in the inflammation associated with atherosclerosis.

It has been noted that high-dose atorvastatin reduces total plasma levels of oxidized phospholipids and immune complexes present on apoB-100, and that after atorvastatin treatment total oxidized phospholipids on all apoB-100 particles was decreased. This lends support to a hypothesis that atorvastatin promotes the clearance of pro-inflammatory, oxidized phospholipids and presumably reduces the immunoreactivity of apoB-100, which may contribute to an observed reduction in ischemic events possibly based on inflammatory reactions after acute coronary syndrome in patients taking this medication.(27)

The components of the invention are designed to increase the efficiency and effectiveness of statins like atorvastatin and consequently will augment the anti-inflammatory benefits of statin use.

Lipoprotein(a)—Lp(a)

An elevated plasma level of Lp(a), represents a major, inherited risk factor for coronary heart disease.(28)

Lp(a) is a modified form of LDL in which a large glycoprotein, apolipoprotein(a) [apo(a)] is covalently bound to apoB-100.(29) The apo(a) protein chain contains five protein domains¹ (30), one of which is homologous with the fibrin-binding domain of plasminogen, a plasma protein that dissolves blood clots (fibrinolysis) when activated. Because of this homology Lp(a) can interfere with fibrinolysis by competing with plasminogen binding.(31;32) It also binds to macrophages, promoting foam cell formation and the deposition of cholesterol in atherosclerotic plaques.(28) ¹A protein domain is a part of protein sequence that can function independently.

Components of this invention affect the impact of Lp(a). The statins and L-carnitine lower Lp(a); l-arginine alters its activity(33) and by implication BH4 may be useful in this regard.

Cholesteryl Ester Transfer Protein (CETP)

CETP inhibitors raise HDL levels in the plasma.(34-36)

CHOL is returned to the liver from peripheral tissues by two pathways: 1) direct delivery by HDL to selective hepatic receptors; 2) transfer of cholesteryl esters from HDL to CETP, thence to LDL or VLDL with subsequent uptake by the liver through hepatic LDL receptors.(34)

Circulating CETP mediates the transfer of cholesteryl esters from HDL to LDL and to triglyceride-rich VLDL. (Triglycerides are simultaneously transferred in the opposite direction.) In the trade-off, HDL cholesteryl ester content is decreased, LDL particles become smaller and denser and VLDL cholesteryl ester is increased. These are unwanted modifications.

Inhibiting CETP is seemed a promising therapeutic target, since drugs that inhibit CETP, such as torcetrapib and anacetrapib, raise HDL levels.(34-36) However, the biochemical modification of CETP is worrisome because of an apparent adverse side-effect of torcetrapib. In a multicenter clinical trial of 15,000 atorvastatin patients with the CETP inhibitor torcetrapib, the latter drug increased the mean particle size of both HDL and of LDL, markedly increased HDL-CHOL levels, and reduced LDL-CHOL. These results occurred both when administered as monotherapy and when administered in combination with a statin.(37) However, the trial was stopped early because, at a mean follow-up of 550 days, torcetrapib therapy was associated with a significant increase in mortality(38) due to both cardiovascular causes and noncardiovascular causes (primarily cancer and infection). The mechanism(s) by which torcetrapib produced these adverse effects is unknown.(38) These problems may be related to global mechanisms of action of this drug class or to molecule-specific adverse effects(39) or may merely have depended upon the lipid profile of the patient.(40) The future of CETP inhibitors is unknown.

Statins and Inflammation/Atherosclerosis

While CHOL is an essential component of cell membranes, when oxidized it can damage cells—via inflammation and atherosclerosis.(41) The pivotal role of inflammation is evident in the initiation, progression and, ultimately, the widespread complications of atherosclerosis.(42) Indeed, atherosclerosis is widely considered to be a chronic inflammatory process, which is related at least in part to excessive levels of reactive oxygen species (ROS) generated by NADPH¹ oxidase (NOX) and progressively inefficient mitochondrial oxidative phosphorylation. ¹NADPH is the reduced form of nicotinamide adenine dinucleotide phosphate; NADP the oxidized form. Mitochondria and the Nox family of NADPH oxidase are major sources of ROS induced by external stimuli (“bad” in this context of this document); . . . but it is important to recognize that ROS are important cell signalers in maintaining cell physiology—an illustrative example; studies suggest that mitochondria generated ROS control NOX redox signaling and that the loss of this controlling cross-talk signal is potentially tumorogenic.

NOX is a multi-component enzyme that generates superoxide anion in the presence of molecular oxygen. It is found in all vascular walls where it represents the major source of the generation of ROS. NOX-mediated superoxide production and subsequent peroxynitrite formation¹ is a principal cause of vascular endothelial dysfunction.(43) Its inhibition suppresses the sequence of cellular events that leads to a variety of cardiovascular diseases, including arteriosclerosis and hypertension. Many antihypertensive agents, angiotensin-converting enzyme (ACE) inhibitors and angiotensin receptor blockers inhibit NOX as do the statins.(44;45) ¹Formation of peroxynitrite in vivo is due to the reaction of the free radical superoxide with the nitric oxide.

Routine mitochondrial respiration is responsible for the production of significant volumes of ROS including superoxide, and its subsequent descendent, peroxynitrite. The latter has a negative effect upon the efficiency of the mitochondrial oxidative energy-producing electron transport chain—adenosine triphosphate (ATP). Although the ATP transport chain is 95% efficient in youth, it significantly declines in efficiency with age. Statin treatment reduces levels of CoQ10, which adversely affects this important energy chain. The invention specifically takes account of this unwanted side effect of statin therapy. (The reduction of this isoprenoid, CoQ10, is probably the underlying basis for the muscle cramping commonly experienced by patients on higher doses of statin therapy. This is discussed in greater detail later.)

There is, however, a useful link between mitochondrial respiration and NOX that provides for the sustained production of ROS—the latter, otherwise undesirable elements, may be important in promoting appropriate programmed cell death (apoptosis).(46) At present there are more questions than answers regarding the association between excessive NOX and mitochondrial-produced ROS in cardiovascular disease. However, the inherent possibilities may provide new treatment opportunities as described in this invention.

NOX exists within what have been called “professional phagocytes” (i.e., neutrophils, eosinophils, monocytes and macrophages). The ROS they produce serve a variety of purposes according to the specific phagocyte. However, within a subendothelial cholesterol-laden macrophage (the foam cell of atherosclerosis) the ROS that is generated is particularly damaging to the vascular endothelium.(47;48)

Components of this invention that modify NOX are tetrahydrobiopterin(49;50), L-arginine(51), and the statins themselves(52-58)—all of these indirectly reduce ROS levels. In addition, the adjuncts alpha-lipoic acid and CoQ10 have significant antioxidant capacity and can directly reduce the ROS that ultimately may be generated by an activated NOX system within or released from mitochondria. The relationships between the statins and these various components, and their physiological attributes are discussed at length below.

A word about the atherogenic inflammatory cytokine tumor necrosis factor alpha (TNF-alpha) is in order here. Patients with rheumatoid arthritis (RA) have a significantly increased risk of developing premature cardiovascular disease. Many similarities have been noticed between the pattern of inflammation in the pathogenesis of atherosclerosis and the mechanisms of inflammation in the pathogenesis of RA.(59) Both of these clinical states share at least one common factor, elevated levels of TNF-alpha. It is noteworthy that an improvement in vascular endothelial function in RA has been demonstrated by treatment with anti-TNF-alpha medications and with statins.(59)

Herein lies another illustration of the potential usefulness of adjunctive statin therapy as represented by the invention—an included component of the invention is D-α-lipoic acid that, like the statins, suppresses the important atherogenic cytokine TNF-alpha. (Discussed in more detail later.)

It is increasingly evident that the mechanisms by which lipid-lowering therapy with statins is beneficial are not entirely explained solely by reductions in serum LDL(60) and that among the non-lipid mechanisms is a reduction in the atherosclerosis-inflammation nexus. In addition, plaque stabilization, reversal of endothelial dysfunction and decreased thrombogenicity should be included in the statin constellation of pleiotropic effects¹.(61;62) (63-65) Because we understand that elevated LDL suppresses constitutive nitric oxide synthetase (eNOS) and propagates a subsequent atherogenic cascade, some of these beneficial mechanisms defined generally as “non-lipid”—should in fact be considered beneficial “lipid” mechanisms. (Statins like many drugs have pleiotropic effects. These effects may be beneficial or undesirable. While both beneficial and undesirable pleiotropy occurs during statin therapy, the former predominate.) ¹The term pleiotropy comes from the Greek word pleion, meaning “more”. In the context of this document it refers to more statin effects than those that are otherwise anticipated or sought after (some good—some bad).

Regardless, inflammation is an important cornerstone of the effectiveness of statin therapy and is addressed by specific anti-inflammatory adjunctive components described by the invention.

Immune System

As introduced in the previous section, there is an emerging understanding that various statin properties modulate the immune system. In cardiovascular disease, the latter exists in conjunction with a poorly regulated cytokine network. While details of the intricate relationship between statins and the immune system (some positive, some negative) are beyond the scope of this document, it may be appropriate to set the stage, since the adjunct components of the invention will be helpful in favorably modulating the immune system in several instances (See infra).

Although a large number of pro- and anti-inflammatory cytokines are of importance, data suggest that the anti-inflammatory cytokine interleukin-10 (IL-10) and the mainly pro-inflammatory cytokines interleukin-6 (IL-6), interleukin-1-beta (IL-1-beta), interleukin-8 (IL-8) and tumor necrosis factor-alpha (TNF-alpha) (See supra) play important roles in the development of cardiovascular disease.(66;66;67)

Relative to the immune system statins act beneficially to: 1) repress T-cell activation, 2) inhibit the expression of specific cell surface receptors on monocytes, 3) inhibit integrin-dependent leukocyte adhesion, 4) decrease IL-6 synthesis (22), and 5) decrease TNF-alpha.(59)

However, on a negative note, statins may stimulate the secretion of caspase-1 protease (cysteine-aspartic acid protease family) that cleaves precursor forms of the inflammatory cytokines IL-1-beta and IL-8 into active peptides. Caspase-1 and related proteases are major mediators of cell death, probably by autoproteolysis.(68-70)

The anti-inflammatory and pro-inflammatory actions of statins in modulating the immune system and inflammatory processes can be modified by the proposed formulations of the invention. This forms the basis for some of the anticipated clinical usefulness expected from the adjunctive regimens described herein.

Endosomes/Lysosome CHOL

As reviewed above, cells acquire CHOL from the blood stream by endocytosis of LDL and VLDL lipoproteins that contain CHOL, cholesteryl esters or both. Once within endosomes, and thence into lysosomes, the esters are hydrolyzed by cholesteryl ester hydrolase into CHOL and fatty acids.

Unlike oxidized-LDL (oxLDL), CHOL esters in non-oxidized LDL are hydrolyzed rapidly to CHOL that, when combined with a fatty acid, is utilized physiologically and which in turn is metabolized to provide ATP. In atherosclerosis, however, excessive levels of oxLDL accumulate in lysosomes increasing local pH, which inhibits the further hydrolysis of cholesteryl esters. After prolonged exposure to oxLDL cholesteryl ester hydrolase is inhibited for a prolonged period and this lysosomal disturbance in turn adversely affects subsequent lipid metabolism.(71) Additionally, these cellular processes are “pushed from upstream” by increased transport of LDL into the cell from the resulting elevated serum LDL. A circular patho-physiology is the result.

Cytosolic CHOL

As the cytosolic cholesteryl esters are hydrolyzed, CHOL accumulates in the cytosol and is transferred to the cell membrane. When the CHOL level in the cell membrane becomes saturated, CHOL “in-transit” to and within the cell membrane is re-esterified and stored in an endosome/lysosome complex for future needs.

Cell Membrane CHOL

When cell membrane CHOL levels are low, trans-membrane movement of LDL into the cytoplasm increases because of an increase in hydrolysis of cholesteryl esters within the lysosomes. This releases more CHOL into the cytoplasm restoring CHOL equilibrium within the cell. In pathologic states like atherosclerosis this process is disturbed. Statins lower serum CHOL and consequently significantly relieve those pathological cellular factors that result from excess LDL as well as improving the statin-based pleiotropic, non-lipid mechanisms referred to above.

The components of this invention have been chosen so that, singly or in the composite, they complement the beneficial lipid and non-lipid effects of the statins and lessen some of the undesirable features.

As alluded to above, beneficial pleiotropic effects of statins include improvement of endothelial dysfunction, an increase in nitric oxide (NO) bioavailability, an increase in antioxidant properties, an inhibition of inflammatory responses, and a stabilization of atherosclerotic plaques.

Individual statins may differ in their pleiotropy and various pleiotropic benefits may occur at different statin dosage levels (often higher) than those required for maximum CHOL reduction.(22;72) The use of adjuncts as described in this invention potentially will support and increase the efficiency of statin therapy by complementing their beneficial pleiotropic characteristics. The reasoning behind this prophetic position has been discussed above and will be expanded below.

Statins and Isoprenoids

Lipid-lowering therapy in patients with hypercholesterolemia has a proven survival benefit for patients with no clinical evidence of coronary disease as well as for patients with established coronary disease. This is true even when serum CHOL concentrations are “normal” or borderline high.

In part this effect is due to the statin-induced inhibition of HMG-CoA reductase within the HMG-mevalonate-isoprenoid-CHOL pathway (See, supra.). As a result of isoprenoid depletion, cytokine production is directly reduced (e.g., pro-inflammatory interleukins (IL-6 and IL-8), proinflammatory prostaglandins and leukotrienes) and the proinflammatory cytokine TNF-alpha is indirectly reduced. And, reduction of some isoprenoids by statins favorably alters monocyte pro-inflammatory functions(67), one of the dominant damaging features of atherosclerosis (See, infra).

However, some useful isoprenoids (e.g., CoQ10, squalene, vitamins A, E, and K) are reduced by statin therapy. The reduction of these isoprenoids can be harmful. Most notable in this regard is CoQ10, the depletion of which can interfere with mitochondrial function leading to clinical myopathy in some patients.

Statins and Nitric Oxide

In the presence of atherosclerosis, statins increase eNOS expression and activity.(73) Secondary to vascular endothelial dysfunction, untreated atherosclerotic results in a decrease in the expression of eNOS. This decrease reduces available NO, a beneficial and strong vasodilator, and this reduction results in a weakening of the inherent counteraction by NO of endothelin-1 (ET-1), a powerful vasoconstrictor. As a result, the physical reduction of the vascular lumen secondary to atherosclerotic thrombosis and endothelial inflammation is complicated by a lumen additionally compromised by unopposed vasoconstriction. Statins, working in a dependent or complementary way with several adjuncts of the invention, will help shift this situation toward the better by improving the NO/ET-1 balance.

To better understand this major anti-vasoconstrictive effect of statins and why it is dependent upon the availability of, and/or augmented by, components of the invention it is important to review the NO-cyclic guanosine 3c,5c-monophosphate (cGMP) generating system of vascular endothelial cells and the important physiologic influence of their resident mitochondria.

Hypercholesterolemia reduces the synthesis and/or bioactivity of endothelial derived NO. However, it also increases the generation of superoxide from NADPH oxidase (See supra) and the generation of superoxide from dysfunctional mitochondria

Under normal conditions, the amino acid l-arginine produces NO via eNOS (utilizing glutathione and activated by the cofactor, tetrahydrobiopterin). The NO thus produced:
Allows for normal vasodilation, which counteracts the vasoconstrictive effects of ET-1 and angiotensin-2;
Reduces platelet adhesion, activation and aggregation—platelets do not normally adhere to nor are they activated by healthy vascular endothelium. However, with reduced NO secondary to hypercholesterolemia, platelets both adhere to and are activated by vascular endothelium. Platelet aggregation mediated by fibrinogen follows. Further platelet activation and aggregation involving thrombin leads to a coagulation cascade and a potentially catastrophic end;
Reduces monocyte (macrophage) endothelial adhesion. Subsequent macrophage migration into the subendothelium where the macrophages accumulate oxidized lipid (to create ‘foam cells’) and distort the endothelial surface, thus starting atherosclerotic and/or thrombotic/embolic processes;
Inhibits the proliferation and migration of vascular smooth muscle cells (VSMC) into the early atherosclerotic lesion. Migration of these muscle cells into the early atherosclerotic lesion is accompanied by a change in VSMC phenotype—from “contractile” type cells into “secretory” type cells. The latter then secrete an extracellular, elastin matrix, which transforms the now progressively maturing lesion into a fibrous plaque. In addition, the migrated VSMC may also become engorged with lipid to form yet more ‘foam cells’. The atherosclerotic lesion thus produced enlarges with the recruitment of more cells, the further elaboration of more extracellular matrix and the accumulation of more lipids. Ultimately it is transformed from a fibrous plaque into a complex plaque. This, the complex plaque, has a fibrous cap that overlies a necrotic core composed of cell debris and esterified/oxidized CHOL; secreted by macrophages;
Reduces atherogenic cytokines—which introduces a non-linear complexity only mentioned here in an abbreviated manner. So-called, “hypercholesterolemic oxidative stress” (HOS) disturbs cell membranes, increasing endothelial permeability (itself a problem), but HOS also probably directly increases the endothelial elaboration of ROS.

Statins and Asymmetric Dimethylarginine (ADMA)

Elevated endothelial and plasma concentration of asymmetric dimethylarginine (ADMA), an endogenous inhibitor of eNOS, is associated with endothelial dysfunction and is prevalent in patients with hypercholesterolemia. Statins decrease hepatic production of cholesterol/cholesteryl esters and LDL effectively reducing hypercholesterolemia, but seem to have limited, if any, influence on reducing the cellular endogenous production of ADMA that occurs in all cells. However, statin's influence in relation to ADMA may become relevant when it is used concomitantly with the NOS substrate, l-arginine. This potential improvement in clinical usefulness is recognized by the invention.

In this regard it is notable that simvastatin used alone does not seem to enhance endothelial function in the presence of elevated ADMA, whereas it does so when ADMA is low. However, a combination of simvastatin and l-arginine can improve endothelial function in the presence of high ADMA.

Statins and Endothelin-1 (ET-1)

ET-1 is an endogenous polypeptide that mediates long-lasting vasoconstriction and plays an important role in the etiology of atherosclerotic vascular disease. Aging and hypertension are two independent cardiovascular risk factors that exhibit increased ET-1 levels with its associated vasomotor dysregulation.

Statins have been shown to reduce ET-1 by inhibiting pre-proET-1 mRNA expression(74) and overall by lessening endothelial dysfunction. L-arginine also has been shown to restore endothelial function and to normalize the synthesis and vasoconstrictor response to ET-1 in hypercholesterolemia.(75;76). This is a desirable pleiotropic effect of the statins with which (74;77;78) l-arginine appears to be complementary. This relationship is taken advantage of by the invention.(75;76;79)

Statins and Prostacyclin (PGI₂)

PGI₂ is a prostanoid, a part of the eicosanoid¹ fatty acid family of signaling molecules—perhaps the most complex in the human body. The family consists of: prostaglandins and leukotrienes (mediators of inflammation); thromboxanes (mediators of vasoconstriction); and prostacyclins, including PGI₂ (vasodilators and inhibitors of platelet aggregation).(80) ¹Eicosanoids are the physiologically active substances derived from arachidonic acid

Healthy vascular endothelial cells release endogenous PGI₂. It reduces platelet aggregation by paracrine signaling via prenylated² protein receptors on nearby platelets and endothelial cells. The prenyl molecule is derived from isoprenoids, and the latter are reduced by statin therapy. ²Prenylation is the addition of a hydrophobic molecule derived from an isoprenoid to a protein, facilitating its attachment to a cell membrane. Prenyl groups are also involved in protein-protein binding via specialized prenyl-binding protein domains.

Because the protein receptor for PGI₂ requires prenylation for activation there has been concern that statins may reduce PGI₂ activity. In a single study this was observed in vitro, but it has not been established in humans.(81)

L-arginine has been observed to inhibit platelet aggregation and reduce pulmonary hypertension. Both of these actions appear similar to parallel actions of PGI₂ and has lead to a theory that it is actually the release of PGI₂ that may be responsible in part for these clinical effects of l-arginine (82;83).

The above may be consistent with the observation that the addition of l-arginine elevates systemic NO elaboration and increases PGI₂ (and the PGI₂/thromboxane ratio), thus beneficially influencing the in vivo homeostasis between vasodilator and vasoconstrictor prostanoids.(84) Whether or not these are cause and effect or clinical correlates, it defines complementarity as represented in the invention. This is of particular relevance given the statin potential for reducing isoprenoid synthesis required for the prenylation (and activation) of the PGI₂ receptor.

3. Components of the Invention

3.A. Statins:
Pravastatin
Lovastatin
Simvastatin
Fluvastatin
Atorvastatin
Rosuvastatin
Pitavastatin

3.B. Adjunctive Components:
Tetrahydrobiopterin (BH4)
L-Arginine
Acetyl L-Carnitine (L-CAR)
Coenzyme Q10 (CoQ10)
D-α-Lipoic Acid (LA)
PDE5 Selective Enzyme Inhibitors

The present invention defines pharmaceutical formulations for use as adjunct oral dosage forms which will increase the effectiveness, efficiency and safety of statins for patients currently using such therapy. The present invention also defines oral dosage forms that will increase the effectiveness, efficiency and safety of an included statin for use as treatment for patients not currently using an HMG-CoA reductase inhibitor. The invention contains specific therapeutic components selected because of their particular and critical combinational physiological actions that complement physiological activities presented by statins in the clinical application of these drugs in treating disorders for which an HMG-CoA reductase inhibitor is appropriate.

One strategy of this invention is to modulate multiple pathophysiological processes to improve the clinical use of statins: This modulation is accomplished by combinations of active ingredients with sometimes disparate, although often complementary or synergistic mechanisms of action, in order to enhance the beneficial actions of statins and reduce the adverse side effects of their use.

3.A. Statins—(Atorvastatin, Pravastatin, Rosuvastatin, Pitavastatin, Simvastatin, Fluvastatin, Lovastatin)

In this document the terms ‘statin’ and ‘HMG-CoA reductase inhibitor’ are used as shorthand to define a member selected from the group consisting of atorvastatin, pravastatin, rosuvastatin, pitavastatin, simvastatin, fluvastatin and lovastatin.

Intensive, i.e., high dose, statin therapy is increasingly recommended to gain full therapeutic (including favorable pleiotropic) value. These doses are often greater and often different than those required for optimum CHOL lowering (See infra). In the context of this invention “full therapeutic value” also means the addition of complementary or synergistic adjunctive components in a formulation designed to maximize the clinical effect of the statins or to permit a clinically necessary reduction of the dose of a statin necessary to achieve a particular therapeutic optimum while reducing unwanted side effects.

The often-stated primary, and not always achievable, goal is to bring all patients with coronary heart disease to an LDL target level below 100 mg/dl or lower. But patients with high vascular risk can benefit from statin therapy regardless of CHOL levels, underlining the importance of assessing the patient's global vascular risk and recognizing the broad nature of statin therapy.(85;86) The mechanisms by which lipid-lowering therapy with statins is beneficial are incompletely explained by focusing attention upon gross serum LDL concentrations at baseline or after treatment.(86-91) The benefits of statin therapy include not only a reduction in the concentration of small, dense LDL—thus shifting the LDL subfractions away from atherogenic LDL—but also a reduction in the level of potentially atherogenic apoB-100.(92;93)

While lipid-lowering therapy in patients with hypercholesterolemia has a proven survival benefit for both primary prevention (i.e., in patients without clinical evidence of coronary disease) and secondary prevention (i.e., in patients with established coronary disease), it is notable that these benefits of treatment can occur even when serum CHOL concentrations are “normal” or borderline high for the given population.

Why is “intensive” statin therapy becoming the gold standard of treatment of coronary artery disease patients who are at high risk?

Treatment to maximally lower LDL levels, when accompanied by significant HDL increases, can cause actual regression of atherosclerosis in coronary disease patients as measured by intravenous ultrasound. This conclusion is from the ASTEROID clinical study: “To Evaluate the Effect of Rosuvastatin on Intravascular Ultrasound-Derived Coronary Atheroma Burden”, which was performed at community and tertiary care centers (349 patients) in the United States, Canada, Europe, and Australia. In this study “very high-intensity statin therapy” (emphasis added) using rosuvastatin 40 mg/d achieved an average LDL of 60.8 mg/dL and increased HDL by 14.7% resulting in significant regression of atherosclerosis.(94)

In a similar vein, compared with moderate lipid lowering with standard-dose statin therapy, intensive lipid lowering with high-dose statin therapy after acute coronary syndromes (ACS) significantly reduces cardiovascular events. In an analysis of >8,600 patients, intensive lipid lowering with high-dose statin therapy after ACS was associated with reduced mortality compared with moderate lipid lowering with standard-dose statin therapy.(95)

While the relationship between intensive statin therapy and subsequent ischemic events is well established, its relationship to the risk of heart failure (HF) after ACS was not well defined until the study, “The Pravastatin or Atorvastatin Evaluation and Infection Trial-Thrombolysis In Myocardial Infarction 22 (PROVE IT-TIMI 22)” study randomized 4,162 patients, stabilized after ACS, to either intensive statin therapy (atorvastatin 80 mg) or moderate statin therapy (pravastatin 40 mg). Treatment with atorvastatin 80 mg significantly reduced the rate of hospitalization for HF, especially in patients with elevated levels of B-type natriuretic peptide¹ (BNP).(96) ¹B-type natriuretic peptide (BNP) is secreted by the heart in response to excessive stretching of heart muscles cells in the left ventricle; notably in congestive heart failure where the BNP level correlates with prognosis in congestive heart failure.

The importance of having multiple pathways available to the practitioner to achieve desirable maximum statin therapy is clear. The present sole choice of merely increasing the dose of a preferred statin, or shifting between statins, is less advantageous than having available rationally defined adjuncts. The adjuncts may improve the effectiveness of statin therapy, permit lower maintenance doses, reduce or avoid side effects and increase prescribing efficiency-B-type certainly the latter will be true if the adjuncts are available complexed with the desired statin. The invention provides important addition therapeutic choices to the clinician.

Effects of Statin Therapy (Probable or Possible):

Statins—Beneficial Effects:
Lower levels of LDL(94)
Regression of atherosclerosis(94)
Plaque stabilization(97)
Reduce vascular inflammation(98;99)
Decrease thrombogenicity(100)
Reverse endothelial dysfunction(101-103)
Decrease ET-1 synthesis(74)
Activate eNOS(104)
Reduce monocyte adhesion to the endothelium(105;106)
Reduce oxidative modification of LDL(107)
Reduce ventricular arrhythmias(108)
Have myocardial anti-ischemic effect(109)
Increase myocardial perfusion(110)

Statin Secondary Benefits—In addition to CHOL lowering and the primary and secondary prevention of cardiovascular disease, statin therapy may be associated with other benefits:
Diabetes—Reduced risk of developing diabetes(111;112)
Hypertension—May lower blood pressure(113;114)
Heart failure—Improves outcome(115;116)
Improvement of erectile dysfunction

Statin Adverse Effects:
Reduces CoQ10 resulting in ↑ oxidation of LDL & ↓ mitochondrial function.(117-120)
Muscle toxicity (cramps, myopathy, including cardiomyopathy)(119;121-125)
Muscle cell death (rhabdomyolysis) in compromised hepatic and renal function(123)
Hepatotoxicity (possibly increased by statin-induced CoQ10 deficiency)(l26-130)

Pleiotropic Effects of Statins

As mentioned above, pleiotropic effects of a drug are actions in addition to those for which the drug was developed—sometimes related to the primary mechanism of action of the drug, sometimes not.(72;86;131) They may constitute undesirable side effects, be neutral or be beneficial. Pleiotropic effects that accompany statin use (in this context, effects that might not obviously be related to lipid reduction) include: improvement of endothelial dysfunction, increased NO bioavailability, production of antioxidant properties, the inhibition of inflammatory responses, decreased thrombogenicity and stabilization of atherosclerotic plaques.(21;86;132) Some of these have previously been referred to in this document.

The pleiotropic effects of all statins are not the same. Precision is elusive. Different dosage schedules, a lack of certainty as to specificity of various anti-inflammatory effects and their relative influence on outcomes, makes relevance . . . well, relative.

Results from various trials raise the possibility that anti-inflammatory activity (and possibly other effects of statins) differ among the statins.(105;133)

That statins modify the inflammatory response is accepted although it is incompletely understood. Statins have multiple intersects within the processes involved in moderating inflammation. These intersections include various actions already alluded to: reduced endothelial dysfunction, improved NO/ET-1 balance (referred to again later), modulation of the platelet aggregation/macrophage vascular adherence/vascular smooth muscle nexus and reductions in superoxide production in vascular cells.

In addition, each statin may have a different direct or indirect influence on mitochondrial function.

In essence we have a circle in a spiral: Unfortunately for expectations of clarity, each circle is continuously being redefined.

The patent anticipates that the components of this invention will augment the favorable pleiotropic actions of statins and lessen the unfavorable ones.

Statins and Regression of Atherosclerosis

As mentioned above, regression of atherosclerotic lesions can occur after lipid lowering without a change in vessel wall thickness or vessel wall area.(134) The benefit of lipid lowering is best illustrated by several observations using coronary angiography. These observations have shown increases in lumen diameter at two and four years, or at least slower progression of vascular stenosis at three years, after the onset of statin therapy.(135;136)

Statins and Plaque Stabilization

Many patients have multiple, unstable plaques in multiple coronary arteries, defining widespread pathology, probably from inflammation in the coronary circulation. Direct surgical intervention aimed at a single lesion is unlikely to be optimal. That statins induce global plaque stabilization is important and probably a reason of substance for the beneficial outcome of statin treatment.

Coronary artery plaque rupture is a central component in most patients with ACS. Statin therapy reduces the rate of progression of plaque development and stabilizes atherosclerotic plaques that have ruptured as well as those that are vulnerable to rupture.(41;97;137;138) One report of 131 patients evaluated the effect of 12 months of atorvastatin therapy using serial three-dimensional volumetric intravascular ultrasound (IVUS).(137;139) Compared to a placebo, atorvastatin reduced the progression of mean plaque volume or thickness (1.2 versus 9.6 mm³) and increased the hyperechogenicity of the plaque, indicating a change in plaque composition from lipid-rich to a fibrotic and calcified status. This change represents increased plaque stability and a reduced tendency for rupture.

Although a variety of factors contribute to the consequences of plaque development, (140;141) an important one seems to be the fibrous integrity of the cap of the plaque. Statin therapy supports the integrity of this fibrous cap and protects against plaque rupture. This benefit appears to be mediated by the inhibition of macrophage proliferation and the promotion of the release of thrombus-promoting tissue factor from macrophages. (41;142;143).

Statins and Inflammation

Elevated serum markers of inflammation, particularly C-reactive protein (CRP): 1) are associated with a progression of atherosclerosis; 2) predict the risk of a first myocardial infarction among apparently healthy men and; 3) are associated with a worse prognosis among patients with stable and unstable angina along with those who have undergone coronary stenting. Statin therapy reduces the serum CRP concentration—an effect that is mostly unrelated to lipid levels at baseline or during therapy.(144-147) The fall in serum CRP begins within 14 days of the start of statin therapy.(146) In support of the predictive potential of CRP; a recent (2008) multicenter trial of apparently healthy persons without hyperlipidemia but with elevated high-sensitivity CRP levels, demonstrated that rosuvastatin significantly reduced the incidence of major cardiovascular events.(148)

Monocyte chemoattractant protein-1 (MCP-1) is essential in atherogenesis. Oxidized lipids regulate the expression of MCP-1 and its release from mononuclear cells. One study investigated: 1) whether statin therapy reduces lipopolysaccharide (LPS)-stimulated MCP-1 production in human whole-blood samples and; 2) the relationship between in vitro LDL oxidation and the production of MCP-1. Statin therapy reduced MCP-1 production in the whole blood of human subjects. These changes appeared to correlate with improvement in the resistance of LDL to oxidation.(149)

Statins and Thrombogenicity

Thrombus formation at the site of plaque rupture accounts for most acute coronary syndromes. Statin therapy has a variety of effects that reduce thrombus formation. (97) (150) Proposed mechanisms include:
Reduced expression of tissue factor in endothelial cells and by macrophages in the atherosclerotic plaque;(100;150;151)
Decreased prothrombin activation and thrombin generation; (151;152)
An improved fibrinolytic profile. (153)

These changes are either independent of, or only partially explained, by CHOL lowering.(150;154) In addition to the above three mechanisms statins also reverse increases in platelet reactivity, platelet-vessel wall interactions, and platelet thrombus formation related to hypercholesterolemia.(155;156)

Statins and Endothelial Dysfunction

Endothelial dysfunction is a dominant feature in atherosclerotic coronary arteries.(157) Endothelial dysfunction permits the induction of vasoconstriction by acetylcholine rather than by NO. (158-164) Acetylcholine causes contraction when applied directly to smooth muscle. And when the endothelial barrier becomes dysfunctional, acetylcholine has direct access to vascular smooth muscle causing vasoconstriction. Studies (165-169) have shown that acetylcholine-induced vasoconstriction can be attenuated or abolished with statin therapy, presumably by restoring the endothelial barrier—an effect that can improve overall vasodilator capacity and measurable myocardial blood flow reserve.(74;170) The improvement in endothelial function can be seen within six weeks from the start of treatment with a HMG-CoA reductase inhibitor.(171) In addition to restoration of the endothelial barrier, there results an increase in endothelial NO activity via restoration of eNOS that had previously been reduced by hypercholesterolemia (See supra). Two beneficial results of increasing the available level of eNOS are: a decrease in ET-1 and a reduced effect of superoxide.(74;104)

Oxidized LDL indirectly diminishes both of these benefits because it downregulates eNOS activity.(74) Thus the importance of maintaining adequate CoQ10 levels to prevent the oxidation of LDL (See infra).

Of additional assistance may be alpha-lipoic acid (AL). Adequate levels of AL appear necessary to stabilize mitochondrial energy levels by increasing the availability of mitochondrial glutathione (GSH), by acting as an antioxidant, and by inhibiting the proinflammatory cytokine, TNF-alpha. (See infra)

Adjuncts that similarly have a beneficial effect upon eNOS and ET-1 have the useful potential to be complementary to statin therapy.

Statins and Reductions in Ventricular Arrhythmias

The major cause of cardiac mortality in patients with CHD is sudden death, primarily due to ventricular tachyarrhythmia. Lipid lowering in patients with CHD reduces the incidence of cardiac death and, among those with an implanted cardioverter-defibrillator, may reduce the rate of life-threatening ventricular arrhythmia (either unstable ventricular tachycardia or ventricular fibrillation).(108;172)

There is evidence that 1-carnitine and CoQ10 may reduce cardiac arrhythmias by reducing mitochondrial dysfunction and improving mitochondrial oxidative phosphorylation.(173-178) It is also suggested that l-arginine may lessen the potential for cardiac arrhythmias induced by myocardial ischemia.(179)

Statins and Erectile Dysfunction (ED)

There is a close relationship between hyperlipidemia/dyslipidemia and ED(180). Endothelial dysfunction is a pathological element common to both.(181) ED and hyperlipidemia/dyslipidemia are both rising in prevalence and there is mounting evidence that these conditions are predictors of cardiovascular disease.

The adjuncts defined by the invention assist the beneficial enhancement of endothelial functions by the statins, such as the reduction of platelet aggregation, the restoration of the NO/ET-1 balance, a lessening of NOX effects, reductions in vascular smooth muscle proliferation and possibly an enhanced regression of atherosclerosis. (See infra)

It is this vascular intersection of the statins, the phosphodiesterase inhibitors and the adjuncts described by the invention (all of which share several physiological fundamentals) that provides a rational basis for considering all three as comprising a complementary group of components that should be evaluated together for their potential usefulness in the clinical world.

Statins: Adverse Effects and Drug Interactions

A variety of statin side effects have been clinically described including headache, nausea, sleep disturbance, elevations in hepatocellular enzymes and alkaline phosphatase, myalgias, myositis, muscle cramps and rhabdomyolysis. These may vary to some extent in probability and severity according to a specific statin and/or dose. As two examples: a group consisting of lovastatin, atorvastatin, rosuvastatin, and simvastatin potentiate warfarin; however, in a similar but different group all but rosuvastatin increase sensitivity to digoxin.

With any drug, keeping dosage at a minimum to achieve a therapeutic endpoint while avoiding toxic side effects is proper. But, as mentioned above, it is currently suggested by many that intense statin dosage (i.e., a dose level pushed beyond mere LDL reduction) is required to obtain maximum long-term cardiovascular benefits: it is within this context of extending statin efficiency and impact, of creating an added-value dosage form, that this invention has been designed.

Statin-induced myopathy is one adverse effect that requires comment. It results at least in part because of statin-induced reductions in CoQ10. This side effect accounts for a recommendation that the statins be used with caution in the elderly, who are predisposed to statin-induced myopathic symptoms. However, this myopathy more broadly reflects the impact of statin-induced (and age-related) reductions of mitochondrial efficiency—said induced reductions result in less vigorous mitochondrial generation of ATP and a consequent production of more mitochondrial superoxide. This invention anticipates formulations designed to improve mitochondrial function by supplying CoQ10, 1-carnitine and AL, which physiologically work in concert and should provide clinical improvement of this class of common myopathies.

3.B. Tetrahydrobiopterin (BH4)

### Oral Dosage Range

Milligrams/day

| | Preferred | Most Preferred |
|---|---|---|
| Tetrahydrobiopterin | 24 to 3000 | 70 to 1200 |

In this document BH4 is used as shorthand for tetrahydrobiopterin in the form of a member selected from the group consisting of 6-lactyl-7′,8′-dihydropterin (sepiapterin), 6-1′,2′-dioxypropyl tetrahydropterin(6-pyruvoyltetrahydropterin), 6-1′-oxo-T-hydroxypropyl tetrahydropterin(6-lactoyltetrahydropterin), and 6-1′-hydroxy-2′-oxypropyl tetrahydropterin (6-hydroxypropy)tetrahydropterin), (6R)-L-erythro-5,6,7,8-tetrahydrobiopterin (BH4), (6R,S)-5,6,7,8-tetrahydrobiopterin, 1′,2′-diacetyl-5,6,7,8-tetrahydrobiopterin sepiapterin, 6-methyl-5,6,7,8-tetrahydropterin 6-hydroxymethyl-5,6,7,8-tetrahydropterin, 6-phenyl-5,6,7,8-tetrahydropterin, and precursors thereof.

Although BH4 is adequately absorbed by the intestine, humans do not obtain sufficient amounts from dietary sources. The body relies on de novo synthesis of BH4 to avoid deficiency.(182)

De novo synthesis of BH4 produces three tetrahydropterins and one dyhydropterin, i.e., 6-lactyl-7′,8′-dihydropterin (sepiapterin). In some circumstances these four molecules have been used with apparent success as stand-ins for BH4. All require sepiapterin reductase to catalyze their reduction to BH4. All are included as alternates to BH4 in this invention. The three tetrahydropterin molecules are:
6-1′,2′-dioxypropyl tetrahydropterin . . . 6-pyruvoyltetrahydropterin (Requires 6-pyruvolytetrahydropterin reductase; then sepiapterin reductase.)
6-1′-oxo-2′-hydroxypropyl tetrahydropterin . . . 6-lactoyltetrahydropterin (Also sometimes referred to as tetrahydrosepiapterin)
6-1′-hydroxy-2′-oxypropyl tetrahydropterin . . . 6-hydroxypropyl tetrahydropterin

BH4 has been clinically investigated as therapy for phenylketonuria (PKC), Parkinson's disease, dystonia, depression, Rett syndrome, infantile autism, senile dementia, Alzheimer's disease, hypertension and atherosclerosis. There have been provocative leads, but except for PKC, the results have been discouraging, possibly because BH4 was used as a monotherapeutic “silver bullet”. While BH4 has broad physiologic involvement and a myriad of chemical interactions, its potential in monotherapy seems limited.

The rational for the presence of BH4 in the invention as a statin adjunct for vascular disease is derived from its function as an obligatory cofactor of the enzyme eNOS, explored below. As previously mentioned, an apparent increase of eNOS is one of the useful pleiotropic side effects of the statins. And, to include another complementary element of the invention, l-arginine is required for eNOS to produce constitutive endothelial NO, which is insufficient in the dysfunctional endothelium of cardiovascular disease (See also “Statins and Nitric Oxide” supra).

BH4/eNOS Relationships and Endothelial Vasoactivity

BH4 is an essential cofactor of eNOS for the production of NO (183), which activates guanylate cyclase; the latter produces cGMP. In turn, cGMP triggers a complex molecular cascade that mediates vasorelaxation through the activation of protein kinases and promotes the subsequent phosphorylation of proteins involved in the regulation of intracellular calcium (Ca2+) levels (e.g., sarcoplasmic Ca2+-ATPase). Commonly, NO is used to represent this cascade.

Endothelial dysfunction associated with atherosclerosis is attributed, at least in part, to alterations in the L-arginine-NO-cGMP pathway and to an excess of ET-1.(74) Such endothelial dysfunction may be improved by BH4 both indirectly in its role as an essential cofactor for eNOS synthesis and directly as a scavenger of oxygen-derived free radicals. Decreased availability of BH4 leads to reduced NO production and an increase in superoxide formation. These two observations support a conclusion that supplementation of BH4 should be considered in the treatment of diseases associated with possible endothelial dysfunction(184) and also support its use as a adjunct component that might therapeutically complement statin therapy. It should also be recalled from a previous section (See “Statins and Nitric Oxide” supra) that hypercholesterolemia, frequently associated with coronary arterial disease, itself reduces eNOS, the level of which is restored by statins—an additional observation that suggests clinical usefulness for a BH4 and statin complex.

BH4 and eNOS Uncoupling

The concept of eNOS uncoupling is important. Molecular oxygen channeled though eNOS ends up as a free radical: either NO (good in appropriate amounts) or superoxide (in this context, always bad). In youth there is adequate l-arginine and BH4, and so the useful free radical NO proceeds down its youthful (physiological) signaling pathway to induce a vasorelaxing cGMP cascade. But in the elderly or in the face of various pathologies there is inadequate l-arginine, BH4 or eNOS and the system “uncouples”: this represents a change for the worse wherein the free radical superoxide becomes the more dominant product and an otherwise youthful, healthy vascular endothelium fades away. And cardiovascular disease ensues.

BH4 and ED

ED is considered a reasonable harbinger of significant cardiovascular disease.(181) This is not surprising.

NO-mediated vascular smooth muscle relaxation occurs in penile erection. In one study, a single dose (200 mg or 500 mg) of BH4 improved the duration of penile rigidity.(185)

Thus, BH4 used alone may be a suitable candidate for the treatment of ED secondary to a variety of causes. It may be expected to be even more effective if combined with the eNOS substrate L-arginine (See the following section) as defined in the invention and/or with one of the phosphodiesterase 5 (PDE5) inhibitors. Furthermore, all three may usefully complement the observed favorable effect of statin therapy upon ED.

3.0 L-Arginine

### Oral Dosage

Milligrams/day

| | Preferred | Most Preferred |
|---|---|---|
| L-Arginine | 75 to 4000 | 250 to 2500 |

In this document L-arginine is defined as a member selected from the group consisting of L-arginine, L-arginine ascorbate, L-arginine citrate or L-arginine and an anion selected from the group consisting of hydroxide, halide or acetate, and derivatives and salts thereof

L-arginine is the obligatory substrate necessary for the normal synthesis of constitutive NO/cGMP required for vasorelaxation. Its clinical use results in an improvement in coronary symptoms, presumably, directly from an increase is available NO and indirectly by beneficially balancing NO/ET-1.

At the risk of being redundant, it is important to emphasize that in the presence of hypercholesterolemia the bioactivity of endothelium-derived NO is reduced and superoxide is generated (See “eNOS Uncoupling” supra), thereby encouraging the development and progression of atherosclerosis. Indeed there are animal studies that suggest that increasing NO by providing l-arginine not only retards the development of atheroma but may cause their regression.(186) An outcome not unlike that observed with the use of the statins.

The concomitant administration of a statin and various selected adjuncts, such as l-arginine—either as separate dosage forms or as part of a single, complexed dosage form—which are potentially complementary in improving NO production while decreasing superoxide production, should be clinically helpful in modulating the progression of atherosclerosis and of other clinical dysfunctions influenced by vascular malfunction, i.e., ED. It may ultimately be found that such adjuncts and their associated or complexed statin are synergetic, not merely complementary.

L-Arginine, Prostacyclin (PGI₂₎ and Platelet Aggregation (See also “Statins and Prostacyclin” supra)

PGI₂ is physiologically released by vascular endothelial cells, inducing vasodilation and reducing platelet aggregation. The PGI₂ signal system for vascular endothelial cells and platelets functions via protein receptors that must be activated by prenylation. The clinical use of statins raises a legitimate worry, since statins reduce isoprenoids and thus reduce the molecules available for prenylation. Clinical data indicate that an “administered therapeutic dose” of atorvastatin does not significantly decrease PGI₂ signaling and does not compromise PGI₂ function in humans.(81) However, there remains a concern as to whether this comforting data can be applied to “all” statins, especially as statin dosage in patients is increased to maximum levels.

This concern is addressed by the components of the invention; for example, not only has in vivo supplementation with l-arginine has been shown to elevate systemic NO as a balance to ET-1(as mentioned above), it also has been shown to increase the PGI₂/Thromboxane A2 (TXA₂) ratio and thus improve the homeostasis between vasodilator (PGI₂) and vasoconstrictor (TXA₂) prostanoids.(187) These dual roles are at least additive if not synergistic and nicely complement the actions of the statins.

In a somewhat different but useful vein, l-arginine reduces human monocyte adhesion to endothelial cells and decreases expression of certain endothelial cell adhesion molecules.(188-190) This action should also clinically complement the observed statin effect of reducing oxidized LDL-induced monocyte adhesion.(169;191)

L-Arginine and ADMA Induced Endothelial Dysfunction (See also “Statins and Asymmetric dimethylarginine (ADMA)” supra).

ADMA is produced endogenously in all human cells and is often elevated in plasma in patients with various diseases, including renal failure, coronary heart disease, hypertension, and diabetes.(192) Chemically related to l-arginine, ADMA competitively inhibits eNOS, which is a probable factor in the pathologies with which ADMA is associated.(193) Its inhibition of eNOS can be offset by l-arginine and this theoretically should further improve endothelial function in those cases where ADMA is elevated.

The effect of statins on plasma ADMA varies, with some statins having no effect (simvastatin(193-196); atorvastatin(197;198)); and some statins causing a decrease in plasma ADMA (fluvastatin(199), rosuvastatin(200) and in a contradictory study, atorvastatin(201)). The variable results may in part relate to statin dosage, duration and undefined conditions that could alter statin pleiotropy. In relation to the latter, consider that by increasing oxidative stress, atherosclerosis alters gene expression, which results in a downregulation of the genetic expression of eNOS and an upregulation of the expression of genes increasing ADMA levels.(202) For this reason any reduction of oxidative stress from any source can be beneficial to in improvements of eNOS and reductions in ADMA. And so the importance of the next paragraph.

While simvastatin has no significant effect on endothelium-dependent vasodilation in patients with high ADMA, when combined with l-arginine there is a notable improvement in NO-dependent vasorelaxation.(203). The impact of l-arginine upon relative ADMA levels when combined with simvastatin was but one factor for the inclusion of this amino acid in the adjunct formulations as proposed by this invention. In this context, also considered was that under conditions of l-arginine or BH4 depletion, eNOS (and neuronal NOS) generate increased superoxide(204). The formulations described in the invention are designed to be functional with all the statins in lessening the endothelial dysfunction associated with hypercholesterolemia and atherosclerosis(205) and in maintaining or increasing statin effectiveness at reduced, or modest statin dosages.

L-Arginine and ED

As previously discussed, when cofactored by BH4 (And further enabled by GSH, see infra) the enzyme eNOS catalyses l-arginine into NO, which activates guanylate cyclase to produce cGMP—a second messenger that leads to vasorelaxation.

The group of phosphodiesterase 5 enzymes degrade cGMP in the corpus cavernosum of the penis, this reduces available NO and this results in less penile rigidity accompanied by symptoms of clinical ED. PDE5 inhibitors block the degradative action of PDE5 upon cGMP in the vascular smooth muscle cells of the corpus cavernosum, thus inducing vasorelaxation, improved penile blood flow and a reduction in clinical ED.

Because l-arginine may also induce prolonged vasorelaxation of human corpus cavernosum by maintaining the endogenous amino acid pool necessary for NO synthesis, l-arginine combined with sildenafil (a PDE5 inhibitor) has been shown to facilitate erections in ED. (206;207) Thus, l-arginine has been proposed as an clinical adjunct for PDE5 inhibitors. It may also act as an even more effective adjunct when complexed with a statin and a PDE5 inhibitor.

The endogenous eNOS inhibitor ADMA is also often elevated in ED, providing an additional pathologic mechanism that may be improved by l-arginine.(207) This relationship was described above.

One final note: Arginase, an enzyme of the liver that catalyzes the hydrolysis of l-arginine to l-ornithine, competes with eNOS for l-arginine. This competition contributes to ED with advanced age. Arginase activity is elevated in aged penile endothelial cells and consequently eNOS activity and cGMP levels are reduced.(208) Thus, it may be argued that supplying older males additional dietary l-arginine as a substrate for eNOS may be clinically useful in alleviating the sexual dysfunction that is common in older males. This is also the usual age cohort of males using statins—for this additional reason having available a PDE5 inhibitor/statin/l-arginine complex makes therapeutic sense.

3.E. Acetyl-L-Carnitine (or Propionyl-L-Carnitine) (L-CAR)

### Oral Dosage

Milligrams/day

| | Preferred | Most Preferred |
|---|---|---|
| L-CAR | 90 to 4000 | 300 to 2500 |

In this document L-CAR is used for Acetyl-L-carnitine in the foi n of a member selected from the group consisting of Acetyl-L-carnitine, propionyl-L-carnitine, L-carnitine ascorbate, L-carnitine citrate or L-carnitine and an anion selected from the group consisting of hydroxide, halide or acetate, and derivatives and salts thereof.

Both acetyl-L-carnitine and propionyl-L-carnitine (L-CAR used for either) are endogenous esters that play a crucial role in cellular fatty acid oxidation in the production of mitochondrial energy derived from long-chain fatty acids.

Once in the cytoplasm, short- and medium-chain fatty acids cross both the outer and inner mitochondrial membranes; these fatty acids subsequently enter the mitochondrial matrix where they undergo β-oxidation. However, because the mitochondrial membrane is not permeable to long-chain fatty acids (which constitute the major source of mitochondrial energy production), a multi-step transportation/oxidation process is required for these long-chain compounds to be capable of providing energy to mitochondria. A detailed description of this highly complex process is beyond the scope of this document except to say that L-CAR is required for this transportation/oxidation process, and therefore for the efficient use of long-chain fatty acids in mitochondrial energy production.

Biopsies of striated muscle in 136 patients with statin-induced myopathies found that in 52% of specimens there were significant biochemical abnormalities in the mitochondrial (or fatty acid) metabolism. Patients have reported variable and persistent muscle symptoms even after the cessation of statin therapy, suggesting that statins induce persistent, long-term abnormalities in striated muscle physiology. This is a worrisome and clinically annoying, side effect of statin use. Muscle symptoms occur in about 7% of treated patients.(209;210)

The physiological effect of statins on cellular energy metabolism, combined with a genetic susceptibility to the triggering of muscle symptoms, may be a nexus that explains the myopathic complaints of many patients.(209) Of course, no modification of genetic influence is possible today, but the invention does address the problem by positing an improvement of mitochondrial oxidative energy levels via the inclusion of L-CAR (and CoQ10 infra) as statin adjuncts. Because L-CAR can function in a complementary manner with CoQ10 in the improvement of mitochondrial (ATP) energy levels, it may be useful in improving statin-induced clinical myopathic symptoms.

In addition to its potential value in avoiding or reducing the severity of statin-induced myopathies by the improvement of cellular fatty acid oxidation, L-CAR has a direct lipid lowering effect. This complements a completely different benefit of statin therapy.

L-CAR and Lipoprotein(a) (Lp(a))

Apolipoproteins are proteins that bind to lipids forming lipoproteins, which as previously noted, transport dietary fats through the bloodstream. In a more global sense, apolipoproteins may also serve as enzyme co-factors and receptor ligands, and by doing so regulate the transport, tissue uptake and metabolism of lipoproteins. However they may become immunoreactive when their LDL phospholipids are oxidized.

Lp(a) is a genetically determined type of plasma LDL in which apoprotein B-100 is linked t o a glycoprotein, apo(a). Elevated Lp(a) levels are a significant cardiovascular risk factor.(211) In one study L-CAR was shown to reduce plasma Lp(a) in patients with otherwise elevated levels.(211)

The additional possible usefulness of L-CAR as an adjunct in cohorts of patients with coronary heart disease (who are frequently also users of the statins) is suggested further by another study in which the effects of the administration of oral L-CAR were compared with a placebo group of patients with suspected acute myocardial infarction. Total cardiac events, including cardiac death and nonfatal infarction, were significantly different—15.6% in the L-CAR treated group vs. 26.0% in the placebo group.(212)

L-CAR and ED

“There is a close link between hyperlipidemia/dyslipidemia and erectile dysfunction (ED), with endothelial dysfunction as a common mechanism. Both ED and hyperlipidemia/dyslipidemia are rising in prevalence with mounting evidence that these conditions are harbingers of cardiovascular disease”.(181)

Raising available L-CAR levels may reduce free fatty acid (FFA)-induced and obesity-associated endothelial dysfunction. This improved endothelial function not only may impact the development of cardiovascular disease, (213) it also may be a useful factor in the treatment of ED.

Because L-CAR is required for the transport and oxidation of mitochondrial fatty acids, as we have seen, it plays a significant role not only in the muscular mitochondrial system, but also in the cardiovascular, nervous, and reproductive systems. The use of L-CAR combined with a PDE5 inhibitor may prove to be more effective than a PDE5 inhibitor used alone in the treatment of ED especially in patients with diabetes. The latter group of patients may be refractory to a PDE5 inhibitor used in monotherapy.(214) In many ways this parallels the potential usefulness of l-arginine for the same clinical condition and is one reason both l-arginine and L-CAR are included as adjuncts in the invention. It may also be true that using l-arginine and L-CAR concurrently as adjuncts, with (or without) a statin, my prove even more beneficial for patients with ED—especially older patients or diabetics with compromised endothelial function who are candidates for therapeutic statins.

Once again, we have an age-related cadre of patients who demonstrate various clinical states that would benefit from adjuncts that function by positively impacting underlying, physiologically-similar problems.

3.D. Coenzyme Q10 (Ubiquinone,Ubiquinol) (CoQ10)

### Oral Dosage

Milligrams/day

| | Preferred | Most Preferred |
|---|---|---|
| Coenzyme Q10 | 5 to 1200 | 15 to 300 |

CoQ10 is frequently referred to as ‘ubiquinone’ (or in its antioxidant, i.e., reduced form, as CoQ₁₀H2 or ubiquinol). It is so named because it is ubiquitous, being in every cell in the human body. It is one of many isoprenoids (including CHOL) that are descendants of the mevalonate pathway: HMG-CoA→mevalonate→isoprenoids (including CHOL, CoQ10, etc). This is the biosynthetic pathway that statins inhibit, thereby reducing the production of CHOL and the other isoprenoids—including, unfortunately, CoQ10.

In the specification and claims of this document CoQ10 is used to represent Coenzyme Q10, ubiquinone, CoQ₁₀H₂ and ubiquinol.

In addition to being reduced in patients treated with statins, CoQ10 levels are also inherently lower in diabetics, in those who smoke and in the elderly.

The mevalonate pathway has multiple branches. The relative impact of each branch is dependent upon cell type. And the relative quantitative activity of each branch is established by DNA-determined, rate-controlling enzymes. It isn't all or none

The mevalonate pathway is dominant in hepatocytes(215) since the liver is the major site of both CHOL and CoQ10 synthesis.(216) CHOL and CoQ10 share available lipoprotein ‘bed space’ after they leave the liver and proceed in the serum to their respective physiological destinations.

By dropping into a colloquial format we might construct a virtual physiological dialogue in order better to understand the vast and complicated constellation of CoQ10 functions. CoQ10 is a ‘jack of all trades’ and apparent master of all of them . . . each depending on the needs of its cellular employer.

Imagine a muscle cell: “I need energy” CoQ10 would reply: “OK, as an essential coenzyme I'll dart into your mitochondria (if I'm not already there) and by enabling beta oxidation, ATP will be produced to satisfy your energy needs. And while there I'll see to it that the oxygen required to make ATP is efficiently burned, avoiding an excess of superoxide”.

Imagine a vascular endothelial cell: “I need protection from free radical damage so I can maintain normal function, continue to constitutively make NO and in a variety of other ways avoid atherosclerosis, but I don't need much ATP”. CoQ10 would reply: “Done.”

Imagine the liver: “I need to satisfy my own ATP energy levels and antioxidant requirements to produce additional CoQ10. CoQ10 would reply: “I'll cover your energy levels and antioxidant needs. In addition I'll ensure that appropriate lipoproteins are manufactured to transport CHOL and triglycerides in the blood stream. I'll ride “side saddle” on these lipoproteins to provide antioxidant defense for them.

CoQ10 as an Antioxidant

In addition to CoQ10's well-established involvement as an integral component of the mitochondrial respiratory chain, it also functions in its reduced form (ubiquinol, remember) as an antioxidant in serum lipoproteins (LDL, VLDL, HDL) protecting these from lipid peroxidation.

CoQ10 is normally present in LDL fragments and inhibits their oxidation.(217) LDL occurs in various subfractions commonly defined by their densities as LDL 1 (most buoyant), LDL2 and LDL3 (least buoyant): LDL3, the densest of the three subfractions has significantly lower protective, integrated levels of CoQ10 and has higher hydroperoxide levels when compared with the lighter counterparts, i.e., there is increased susceptibility to oxidation in the LDL3 subfraction.(10, 11) It is notable, and possibly causative, that the LDL3 subfraction is more abundant in individuals having coronary heart disease than in healthy and normal subjects.(12, 13)

After CoQ10 supplementation all three LDL subfractions may display increased CoQ10 levels, but LDL3 has the greatest CoQ10 increase. There is an associated significant decrease in hydroperoxide levels. Presumably one can infer a reduced the risk of coronary heart disease.(218;219) To simplify: The most damaging, dangerous LDL is the subfraction most affected by dietary CoQ10 supplementation.

Oxidative events can occur in many places, such as the vascular subendothelial space where oxLDL contributes to the atherogenic process by altering the chemotaxis of monocytes (and of monocyte-derived macrophages) that ingest the oxidatively-modified LDL particles through a scavenger receptor-mediated mechanism. Microscopically these macrophages have a foamy appearance because of the large number of vesicles that accumulate within their cytoplasm. Vascular subendothelial collections of these foam cells represent the “fatty streak”, which is the first grossly visible lesion in the development of atherosclerosis. However, as described above, antioxidants such as CoQ10 reduce the propensity of LDL to oxidize—specifically, CoQ10 may protect the particles of lipoprotein from oxidation more efficiently than vitamin E.(8, 9) Not surprisingly, a reduced ratio of CoQ10/LDL can be viewed as a risk factor for cardiovascular disease, arteriosclerosis and the erectile dysfunction that is a frequent symptom of this group of patients.(220)

The principal complication of type 2 diabetes is cardiovascular disease—80% of patients with diabetes will develop or die of some type of major vascular event (about half of whom become sexually dysfunctional along the way). The American College of Physicians recommends moderate doses of statins for people with diabetes since, at least in patients with type 2 diabetes, this clearly reduces cardiovascular risk.(221)

Unfortunately, as stated above, CoQ10 is inherently and especially reduced in diabetics, perhaps because of the increase in free radicals in this disease and the resulting overutilization of antioxidant defenses. This is of singular importance since most diabetics eventually require statin therapy. Unfortunately the introduction of statins further reduces already lower CoQ10 levels: this emphasizes the importance, especially in this group of patients, of the availability of adjunctive CoQ10 supplementation either complexed with a statin or administered concurrently but separately from a statin.

It is notable that diabetes is the most common cause of sexual dysfunction. This problem exists in diabetics because elevated levels of oxLDL downregulates eNOS activity.(222) The fact that CoQ10 reduces the oxidation of LDL thus preserving eNOS, thus reducing vascular endothelial dysfunction in statin-using diabetic patients, presents an argument for the clinical use of CoQ10 as adjunct therapy for ED associated with diabetics, especially those using statins.

It should be recalled that BH4 and l-arginine also participate in protecting adequate levels of eNOS. Once again we find complementarity between these three elements (CoQ10, l-arginine, BH4) and have the opportunity to use them in various combinations in formulations designed as adjuncts for use in that cohort of patients frequently associated with or actively using statins.

CoQ10 in Mitochondrial Bioenergetics: Electron Transport Chain

Mitochondrial bioenergetics defines the oxidative phosphorylation process that produces energy (ATP). The respiratory electron transport chain is composed of multiple subunit complexes linked by mobile electron carriers. Notably, one of these is CoQ10. The flow of electrons releases energy that is used in the phosphorylation of ADP to ATP.

CoQ10 is present in all human cells and in cooperation with multiple substances, including L-CAR, is responsible for ninety-five percent of all the human body's energy requirements (ATP). Those organs with the highest energy requirements—such as the heart, the lungs, and the liver—have the highest concentration of CoQ10.(223;224)

Clinically, chronic heart failure (CHF) is a common end result of coronary artery disease, especially in diabetics. In CHF the heart does not have enough mitochondrial-produced energy. Low plasma levels of CoQ10 may be an independent predictor of CHF mortality.(225;226)

As stated previously, endogenous CoQ10 production is reduced in patients treated with statins.(118) While statins are well established in the treatment of coronary artery disease, their role in the treatment of CHF has been controversial since beneficial as well as possible harmful side effects may occur.(227)

Along similar lines there has been a recommendation that statins be used with caution in the very elderly who are predisposed to statin-induced myopathic symptoms secondary to reduced CoQ10. These symptoms may also simply be age-related to a ‘normal’ reduced mitochondrial efficiency resulting in less mitochondrial generation of ATP and a consequent accumulation of mitochondrial superoxide.

This invention is designed to improve mitochondrial function in the statin-using patient cadre (which also includes a high volume of those afflicted by ED) by supplying CoQ10, L-CAR and alpha-lipoic acid adjuncts, which work in concert. (D-alpha-lipoic acid is discussed next)

3.F. D-Alpha-Lipoic Acid (LA)

### Oral Dosage

Milligrams/day

| | Preferred | Most Preferred |
|---|---|---|
| D-α-Lipoic Acid | 30 to 1500 | 75 to 600 |

In this document LA is used to define a member selected from the group consisting of alpha-lipoic acid, D-Alpha-lipoic acid, D-α-Lipoic Acid, dihydrolipoic acid and derivatives and salts thereof.

Alpha-lipoic acid (1,2-dithiolane-3-pentanoic acid) is a proglutathione metabolic antioxidant. LA is a low molecular weight substance that occurs naturally in the R(+) configuration.(228;229) It is both water and lipid-soluble (It is amphipathic because of the hydrophilic carboxylic acid group at the end of its hydrocarbon side chain.) and crosses the blood-brain barrier. Within cells and tissues, the salt form (alpha-lipoate) is reduced to dihydrolipoate that ultimately is exported to the extracellular medium; hence, it provides antioxidant protection in both extracellular and intracellular, lipid and aqueous environments.

LA is necessary for: The production of NO via eNOS, increasing levels of reduced GSH, improving insulin sensitivity (by reducing insulin resistance), and for the inhibition of the pro-inflammatory cytokine TNF-alpha and its activation of NF-kappaB (See infra). In short, its metabolic activities suggest how it may usefully function in multiple synergistic or complementary ways with the statins and with other components of this invention already described.

While ROS (e.g., superoxide anion and hydrogen peroxide) act as second messengers of intracellular signaling, NF-kappa B acts as a redox-sensitive transcription factor and plays a key role in the pathogenesis of atherosclerosis. However, LA inhibits NF-kappaB activation in vitro and ex-vivo.(230-233) This possibly presents an explanation for the very efficient antioxidant properties of LA. The fact that statins in vitro diminish NF-kappa B activation(234-237) elicited by oxidative stress may define an additional molecular usefulness that they may contribute to the clinical treatment of cardiovascular disease.(238) As a statin adjunct, LA may further complement this useful trait shared with the HMG-CoA reductase inhibitors. And once again it may be useful in reducing symptoms of ED commonly present in the cardiovascular pathologies.

LA can be effectively administered as a dietary supplement.(230)

LA, GSH and Other Antioxidant Activities

The reduced form of GSH is the most important cellular and mitochondrial antioxidant (it is a powerful scavenger of superoxide, hydrogen peroxide, OH⁻, HOCl, NO^{•} and lipid peroxides) and is an essential cofactor for the ability of eNOS to synthesize NO from LA. Reduced levels of GSH hamper endothelial cell functions. Among other causes, low GSH levels may follow an increase of free radical production (e.g., hyperglycemia-induced) and/or to a decrease of NADPH levels (inter alia in diabetes mellitus).

GSH is a sulfur-containing tripeptide composed of cysteine, glycine, and glutamic acid. It participates in the hepatic detoxification of many compounds via glutathione S-transferase. GSH is a component of various antioxidant enzyme systems, not only the aforementioned glutathione S-transferase, but also as a glutathione peroxidase that reduces lipid hydroperoxides to their corresponding alcohols and then to water. Thus, in various ways GSH, especially via a reduction in ROS it lessens oxidative damage, particularly in mitochondria.

GSH is essential for mitochondrial function. That the net efflux of GSH from mitochondria is very slow suggests that this transport mechanism functions to conserve mitochondrial GSH especially during periods of cytoplasmic GSH depletion.(239)

The mitochondria do not synthesize GSH (mitochondria do not have the enzyme required for such synthesis)(239), but rather it enters mitochondria from the cytoplasm, via a process characterized by a slow net transport system and a more rapid exchange transport pathway.

Conventional wisdom is that orally administered GSH is “destroyed” during its first pass through the liver and is not practical for dietary supplemental delivery. There is a contrary view that believes that oral supplementation of GSH may be useful to enhance tissue availability.(240) But it remains uncertain how efficient the absorption of GSH actually is. There is agreement that the absorption of LA is efficiently adequate.

Both the alpha-lipoate (oxidized) and dihydrolipoate (reduced) forms of LA are potent antioxidants, which are capable of acting directly against ROS activity. Furthermore either the oxidized or the reduced forms can regenerate other antioxidants (e.g., ascorbic acid, α-tocopherol) through redox cycling, and can thus directly and indirectly raise intracellular GSH levels by modulating ROS that adversely impact GSH production. (241)

LA in Diabetes

Diabetes is associated with oxidative stress and hyperglycemia lowers GSH levels. These low levels can be restored by LA. The presumption is that this occurs via a reduction of the excessive levels of ROS commonly present in diabetes, which results in an increased availability of GSH.(242;243) Diabetics also have increased concentrations of inflammatory cytokines such as TNF-alpha, which are also reduced by LA.(232;233;244;245) Curiously, in rats, the beneficial effects of simvastatin may be related directly to the reduction of this inflammatory cytokine, TNF-alpha.(246) In fact multiple studies have now demonstrated that statins inhibit NF-kappaB activity.(234-237). In this regard we see LA and the statins working in a complementary fashion.

Diabetic neuropathy is a common chronic complication of diabetes. The neuropathies are numerous and include: 1) peripheral neuropathies (e.g., pain, paresthesia, numbness), and 2) autonomic neuropathies (e.g., tachycardia at rest, myocardial ischemia, postural hypotension and sudden death, deterioration of gastric evacuation, diabetic diarrhea or constipation, ED, impaired function of the sweat glands, impaired pupillary reaction).

If we recall the pleiotropic benefits of statin use re: the reduction of cellular oxidative stress, (not repeated here since this has been covered extensively earlier in this document) it comes as no surprise that statins are useful in the treatment of diabetic neuropathy. (247-252) And since these statin benefits appear to parallel those of LA, we may have once again established an important adjunctive relationship defined by this invention.

LA in ED

As stated previously the reduced form of GSH is an important cofactor (as is BH4) for eNOS in the synthesis of NO from l-arginine. A reduced level of GSH, due to several factors (such as a hyperglycemia-induced increase of free radical production, or to a decrease of NADPH levels as usually exists in advanced age, diabetes mellitus and the metabolic syndrome) may play an important role in the etiology of ED. The red blood cell concentration of GSH has been found to be significantly lower in patients with ED than in controls. In particular, GSH is significantly lower in patients with ED and diabetes mellitus compared to patients with ED, but without diabetes mellitus. In fact, a negative correlation seems to exist not only between GSH and fasting glucose concentrations, but also with the duration of diabetes. This most probably occurs because GSH depletion can lead to a reduction of NO synthesis, thus impairing vasodilatation in the corpora cavernosum(253). LA improves the NO-mediated endothelium-dependent relaxation of corpus cavernosum, similar to L-CAR, CoQ10 and BH4.(180;181;181;185;253-255) A formulation containing various combinations of these elements—LA, L-CAR, CoQ10, BH4 may prove to be clinically useful for ED. They also can be formulated to serve as adjuncts for patients using statins. The formulations contemplated by the invention incorporate all of these as active ingredients in various combinations.

A reminder: Any significant slowing of cellular ATP energy formation and of electron transport usually results in endothelial dysfunction in blood vessels and contributes to the development of a variety of vascular complications. This may explain why antioxidants like vitamin E, which work by scavenging already formed toxic oxidation products, have failed to show beneficial effects on vascular dysfunction. It suggests why it is that antioxidants like L-CAR and LA, which work as intracellular superoxide scavengers early in the process, appear to improve mitochondrial function better and reduce DNA damage more effectively.(256) This is especially true in diabetes and aging, where early on, oxidative stress plays a key role in the pathogenesis of vascular complications of which an initial step is the development of endothelial dysfunction.

3.G. Phosphodiesterase 5 (PDE5) Selective Enzyme Inhibitors

In this document PDE5 is used to define a selective phosphodiesterase 5 inhibitor selected from the group consisting of sildenafil, tadalafil, vardenafil, udenafil, avanafil or benzamidenafil.

PDE inhibitors (specifically, PDE5 selective enzyme inhibitors are referred to in this document—see below) prevent the enzymatic (hydrolytic) degradation of cyclic guanosine monophosphate (cGMP), augmenting and prolonging the cGMP effect. cGMP is a second messenger¹ that has numerous functions including relaxing smooth muscle cells leading to vasodilation, which in the corpora cavernosum of the penis results in an erection. ¹A second messenger is an intermediary molecule that is generated as a consequence of hormone-receptor interaction, which, in turn, proceeds to activate effector proteins within the cell causing a cellular response.

| | Milligrams/day |
|---|---|
| | Most Preferred |
| Sildenafil | 25 to 100 |
| Tadalafil | 2.5 to 20 |
| Vardenafil | 5 to 20 |
| Udenafil | 100 to 200 |
| | (Korea) |
| Avanafil | Not Available (5 year estimate) |
| Benzamidenafil | Not Available (257) |

Components of the invention: l-arginine, BH4, L-CAR, LA—and statins should all work in concert to increase cyclic GMP and thereby enhance the effect of PDE5 enzyme inhibitors. Details relating to each component in this regard are found above in their respective component sections.

Compositions, Formulations, and Dosages

In general, the dosage forms of this invention contemplates the use powders, liquids, emulsions, immediate release tablets, sustained releases tablets, capsules, transmembrane delivery systems, electrophoretic delivery systems and other clinically effective forms of delivery.

The dosage forms of this invention can be formulated for administration at rates of one or more unit dosage forms per day, or one or more unit dosage forms at intervals longer than one day.

A. Single-Layer Tablets

In certain embodiments of the invention, the oral dosage form is a substantially homogeneous single layer tablet that releases all of its components into the stomach upon ingestion.

Oral unit dosage forms to be taken three to four times per day for immediate release tablets are preferred.

B. Sustained-Release Tablets

In certain other embodiments of the invention, the oral dosage form is a tablet in which the active agents are protected by an acid-resistant coating for release only in the intestine, and optionally in a sustained-release manner over a period of time.

The polymer matrix of the controlled (sustained) release tablet, having been given an enteric coating in the granulation process with EUDRAGIT, does not dissolve in the acid pH of the stomach, but remains intact until it passes to the upper part of the small intestine, where the enteric coating dissolves in the more alkaline environment of the intestine. The polymeric matrix then immediately begins to imbibe water from the intestinal fluid, forming a water-swollen gel. The agents incorporated into this layer are then available for intestinal absorption as they osmotically diffuse from the gel. The rate of diffusion the agent is reasonably constant for the useful life of the matrix (approximately four hours), by which time the incorporated agent is finally depleted and the matrix disintegrates. Such a single layer controlled release tablet, substantially homogenous in composition, is prepared as illustrated in the examples that follow.

The slower, more sustained release of the active agents can be achieved by placing the active agents in one or more delivery vehicles that inherently retard the release rate. Examples of such delivery vehicles are polymeric matrices that maintain their structural integrity for a period of time prior to dissolving, or that resist dissolving in the stomach but are readily made available in the post-gastric environment by the alkalinity of the intestine, or by the action of metabolites and enzymes that are present only in the intestine. The preparation and use of polymeric matrices designed for sustained drug release is well known. Examples are disclosed in U.S. Pat. No. 5,238,714 (Aug. 24, 1993) to Wallace et al.; Bechtel, W., Radiology 161: 601-604 (1986); and Tice et al., EPO 0302582, Feb. 8, 1989. Selection of the most appropriate polymeric matrix for a particular formulation can be governed by the intended use of the formulation. Preferred polymeric matrices are hydrophilic, water-swellable polymers such as hydroxymethylcellulose, hydroxypropylcellulose, hydroxyethylcellulose, hydroxymethylpropylcellulose, polyethylene oxide, and porous bioerodible particles prepared from alginate and chitosan that have been ionically crosslinked.

A delayed, post-gastric, prolonged release of the active ingredients in the small intestine (duodenum, ileum, jejunum) can also be achieved by encasing the active agents, or by encasing hydrophilic, water-swellable polymers containing the active agents, in an enteric (acid-resistant) film. One class of acid-resistant agents suitable for this purpose is that disclosed in Eury et al., U.S. Pat. No. 5,316,774 (“Blocked Polymeric Particles Having Internal Pore Networks for Delivering Active Substances to Selected Environments”). The formulations disclosed in this patent consist of porous particles whose pores contain an active ingredient and a polymer acting as a blocking agent that degrades and releases the active ingredient upon exposure to either low or high pH or to changes in ionic strength. The most effective enteric materials include polyacids having a pKₐ of from about 3 to 5. Examples of such materials are fatty acid mixtures, methacrylic acid polymers and copolymers, ethyl cellulose, and cellulose

The slower, more sustained release of the active agents can be achieved by placing the active agents in one or more delivery vehicles that inherently retard the release rate. Examples of such delivery vehicles are polymeric matrices that maintain their structural integrity for a acetate phthalates. Specific examples are methacrylic acid copolymers sold under the name EUDRAGIT®, available from Rohm Tech, Inc., Maiden, Mass., USA; and the cellulose acetate phthalate latex AQUATERIC®, available from FMC Corporation, New York, N.Y., USA, and similar products available from Eastman-Kodak Co., Rochester, N.Y., USA.

Acid-resistant films of these types are particularly useful in confining the release of active agents to the post-gastric environment. Acid-resistant films can be applied as coatings over individual particles of the components of the formulation, with the coated particles then optionally compressed into tablets. An acid-resistant film can also be applied as a layer encasing an entire tablet or a portion of a tablet where each tablet is a single unit dosage form.

The dosage forms of the invention optionally include one or more suitable and pharmaceutically acceptable excipients, such as ethyl cellulose, cellulose acetate phthalates, mannitol, lactose, starch, magnesium stearate, sodium saccharin, talcum, glucose, sucrose, carbonate, and the like. These excipients serve a variety of functions, as indicated above, as carriers, vehicles, diluents, binders, and other formulating aids.

Oral unit dosage forms to be taken once or four times daily for controlled (sustained) release tablets are preferred.

The amounts of the primary components of the dosage forms of the pharmaceutical preparation of this invention can vary. Expressed in terms of milligrams per day some examples of components and preferred ranges are illustrated in the following Examples.

However, the following Examples are used for illustrative purposes and do not encompass the entirety of the formulations contemplated by the invention, i.e., they are not intended to limit the variety of formulation combinations contemplated by the invention.

Examples of possible formulations are:

Example 1 Statin Adjunct Complex: Pravastatin

Example 2 Statin Adjunct BT (Bed Time)

Example 3 Statin Adjunct CVS (Cardiovascular Support)

Example 4 Statin Adjunct ED (Erectile Dysfunction)

### EXAMPLE 1

### Pravastatin+ChronoRX Complex

### EXAMPLE 2

### Statin Adjunct BT (Bedtime)—Statin Administered Separately

### EXAMPLE 3

### Cardiovascular Support—Statin Administered Separately

### EXAMPLE 4

### Treatment of Erectile Dysfunction

A statin, if used, is either administered separately or may be included in the formulation.

In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is not excluded from the scope of the claim. All patents, patent applications, and other published reference materials cited in this specification are hereby incorporated herein by reference in their entirety. Any discrepancy between any reference material cited herein or any prior art in general and an explicit teaching of this specification is intended to be resolved in favor of the teaching in this specification. This includes any discrepancy between an art-understood definition of a word or phrase and a definition explicitly provided in this specification of the same word or phrase.

### REFERENCE LIST

(1) Abramson J, Wright J M. Are lipid-lowering guidelines evidence-based? Lancet. 2007; 369:168-69.

Ref ID: 107540
(2) Kendrick J S, Chan L, Higgins J A. Superior role of apolipoprotein B48 over apolipoprotein B100 in chylomicron assembly and fat absorption: an investigation of apobec-1 knock-out and wild-type mice. Biochem J. 2001; 356:821-27.

Ref ID: 108453
(3) Werner A, Havinga R, Perton F, Kuipers F, Verkade H J. Lymphatic chylomicron size is inversely related to biliary phospholipid secretion in mice. Am J Physiol Gastrointest Liver Physiol. 2006; 290:G1177-G1185.

Ref ID: 108316
(4) Wong J, Quinn C M, Guillemin G, Brown A J. Primary human astrocytes produce 24(S),25-epoxycholesterol with implications for brain cholesterol homeostasis. J Neurochem. 2007; 103:1764-73.

Ref ID: 108213
(5) Sagin F G, Sozmen E Y. Lipids as key players in Alzheimer disease: alterations in metabolism and genetics. Curr Alzheimer Res. 2008; 5:4-14.

Ref ID: 108304
(6) Guyton A C, Hall J E. Textbook of medical physiology. 11th ed ed. Philadelphia: Elsevier Saunders; 2006.

Ref ID: 108284
(7) Ferguson E, Singh R J, Hogg N, Kalyanaraman B. The mechanism of apolipoprotein B-100 thiol depletion during oxidative modification of low-density lipoprotein. Arch Biochem Biophys. 1997; 341:287-94.

Ref ID: 108297
(8) Matsukawa N, Nariyama Y, Hashimoto R, Kojo S. Higher reactivity of apolipoprotein B-100 and alpha-tocopherol compared to sialic acid moiety of low-density lipoprotein (LDL) in radical reaction. Bioorg Med Chem. 2003; 11:4009-13.

Ref ID: 108294
(9) Obama T, Kato R, Masuda Y, Takahashi K, Aiuchi T, Itabe H. Analysis of modified apolipoprotein B-100 structures formed in oxidized low-density lipoprotein using LC-MS/MS. Proteomics. 2007; 7:2132-41.

Ref ID: 108290
(10) Schiopu A, Bengtsson J, Soderberg I, Janciauskiene S, Lindgren S, Ares M P et al. Recombinant human antibodies against aldehyde-modified apolipoprotein B-100 peptide sequences inhibit atherosclerosis. Circulation. 2004; 110:2047-52.

Ref ID: 108293
(11) Hashimoto R, Matsukawa N, Nariyama Y, Ogiri Y, Hamagawa E, Tanaka K et al. Evaluation of apolipoprotein B-100 fragmentation and cross-linkage in serum as an index of atherosclerosis. Biochim Biophys Acta. 2002; 1584:123-28.

Ref ID: 108295
(12) Valentinova N V, Gu Z W, Yang M, Yanushevskaya E V, Antonov I V, Guyton J R et al. Immunoreactivity of apolipoprotein B-100 in oxidatively modified low density lipoprotein. Biol Chem Hoppe Seyler. 1994; 375:651-58.

Ref ID: 108298
(13) Leitinger N. Cholesteryl ester oxidation products in atherosclerosis. Mol Aspects Med. 2003; 24:239-50.

Ref ID: 108299
(14) Hoppe G, Ravandi A, Herrera D, Kuksis A, Hoff H F. Oxidation products of cholesteryl linoleate are resistant to hydrolysis in macrophages, form complexes with proteins, and are present in human atherosclerotic lesions. J Lipid Res. 1997; 38:1347-60.

Ref ID: 108300
(15) Spickett C M, Rennie N, Winter H, Zambonin L, Landi L, Jerlich A et al. Detection of phospholipid oxidation in oxidatively stressed cells by reversed-phase HPLC coupled with positive-ionization electrospray [correction of electroscopy] MS. Biochem J. 2001; 355:449-57.

Ref ID: 108286
(16) Halliwell B. The role of oxygen radicals in human disease, with particular reference to the vascular system. Haemostasis. 1993; 23 Suppl 1:118-26.

Ref ID: 6127
(17) Stryer L. Biochemistry. 4th ed ed. New York: W. H. Freeman; 1995.

Ref ID: 108209
(18) Yamane K, Kataoka S, Le N A, Paidi M, Howard W J, Hannah J S et al. Binding affinity and particle size of LDL in subjects with moderate hypercholesterolemia: relationship with in vivo LDL metabolism. J Lipid Res. 1996; 37:1646-54.

Ref ID: 108314
(19) Moriyama T, Wada M, Urade R, Kito M, Katunuma N, Ogawa T et al. 3-hydroxy-3-methylglutaryl coenzyme A reductase is sterol-dependently cleaved by cathepsin L-type cysteine protease in the isolated endoplasmic reticulum. Arch Biochem Biophys. 2001; 386:205-12.

Ref ID: 107965
(20) Greger R, Windhorst U. Comprehensive human physiology from cellular mechanisms to integration. Berlin: Springer; 1996.

Ref ID: 107979
(21) Vaughan C J, Gotto A M, Jr., Basson C T. The evolving role of statins in the management of atherosclerosis. J Am Coll Cardiol. 2000; 35:1-10.

Ref ID: 107363
(22) Crisby M. Modulation of the inflammatory process by statins. Timely Top Med Cardiovasc Dis. 2005; 9:E3.

Ref ID: 107606
(23) Pignataro O P, Radicella J P, Calvo J C, Charreau E H. Mitochondrial biosynthesis of cholesterol in Leydig cells from rat testis. Mol Cell Endocrinol. 1983; 33:53-67.

Ref ID: 108872
(24) Leon C, Hill J S, Wasan K M. Potential role of acyl-coenzyme A:cholesterol transferase (ACAT) Inhibitors as hypolipidemic and antiatherosclerosis drugs. Pharm Res. 2005; 22:1578-88.

Ref ID: 108332
(25) Genest J, Jr., McNamara J R, Ordovas J M, Jenner J L, Silberman S R, Anderson K M et al. Lipoprotein cholesterol, apolipoprotein A-I and B and lipoprotein (a) abnormalities in men with premature coronary artery disease. J Am Coll Cardiol. 1992; 19:792-802.

Ref ID: 108873
(26) Yang C, Gu Z W, Yang M, Gotto A M, Jr. Primary structure of apoB-100. Chem Phys Lipids. 1994; 67-68:99-104.

Ref ID: 108334
(27) Tsimikas S, Witztum J L, Miller E R, Sasiela W J, Szarek M, Olsson A G et al. High-dose atorvastatin reduces total plasma levels of oxidized phospholipids and immune complexes present on apolipoprotein B-100 in patients with acute coronary syndromes in the MIRACL trial. Circulation. 2004; 110:1406-12.

Ref ID: 108333
(28) Zioncheck T F, Powell L M, Rice G C, Eaton D L, Lawn R M. Interaction of recombinant apolipoprotein(a) and lipoprotein(a) with macrophages. J Clin Invest. 1991; 87:767-71.

Ref ID: 108755
(29) Steyrer E, Durovic S, Frank S, Giessauf W, Burger A, Dieplinger H et al. The role of lecithin: cholesterol acyltransferase for lipoprotein (a) assembly. Structural integrity of low density lipoproteins is a prerequisite for Lp(a) formation in human plasma. J Clin Invest. 1994; 94:2330-2340.

Ref ID: 108751
(30) McLean J W, Tomlinson J E, Kuang W J, Eaton D L, Chen E Y, Fless G M et al. cDNA sequence of human apolipoprotein(a) is homologous to plasminogen. Nature. 1987; 330:132-37.

Ref ID: 108752
(31) Loscalzo J, Weinfeld M, Fless G M, Scanu A M. Lipoprotein(a), fibrin binding, and plasminogen activation. Arteriosclerosis. 1990; 10:240-245.

Ref ID: 108754
(32) Palabrica T M, Liu A C, Aronovitz M J, Furie B, Lawn R M, Furie B C. Antifibrinolytic activity of apolipoprotein(a) in vivo: human apolipoprotein(a) transgenic mice are resistant to tissue plasminogen activator-mediated thrombolysis. Nat Med. 1995; 1:256-59.

Ref ID: 108753
(33) Schlaich M P, John S, Langenfeld M R, Lackner K J, Schmitz G, Schmieder R E. Does lipoprotein(a) impair endothelial function? J Am Coll Cardiol. 1998; 31:359-65.

Ref ID: 108749
(34) Brewer H B, Jr. Increasing HDL Cholesterol Levels. N Engl J Med. 2004; 350:1491-94.

Ref ID: 108560
(35) Brousseau M E, Schaefer E J, Wolfe M L, Bloedon L T, Digenio A G, Clark R W et al. Effects of an inhibitor of cholesteryl ester transfer protein on HDL cholesterol. N Engl J Med. 2004; 350:1505-15.

Ref ID: 108562
(36) Forrester J S, Makkar R, Shah P K. Increasing high-density lipoprotein cholesterol in dyslipidemia by cholesteryl ester transfer protein inhibition: an update for clinicians. Circulation. 2005; 111:1847-54.

Ref ID: 108561
(37) Brousseau M E, Schaefer E J, Wolfe M L, Bloedon L T, Digenio A G, Clark R W et al. Effects of an inhibitor of cholesteryl ester transfer protein on HDL cholesterol. N Engl J Med. 2004; 350:1505-15.

Ref ID: 108349
(38) Barter P J, Caulfield M, Eriksson M, Grundy S M, Kastelein J J, Komajda M et al. Effects of torcetrapib in patients at high risk for coronary events. N Engl J Med. 2007; 357:2109-22.

Ref ID: 108350
(39) Nissen S E, Tardif J C, Nicholls S J, Revkin J H, Shear C L, Duggan W T et al. Effect of torcetrapib on the progression of coronary atherosclerosis. N Engl J Med. 2007; 356:1304-16.

Ref ID: 108352
(40) Rensen P C, Jukema J W. [Torcetrapib increases mortality in patients with a high risk of cardiovascular disorders]. Ned Tijdschr Geneeskd. 2008; 152:1088-90.

Ref ID: 108559
(41) Crisby M, Nordin-Fredriksson G, Shah P K, Yano J, Zhu J, Nilsson J. Pravastatin treatment increases collagen content and decreases lipid content, inflammation, metalloproteinases, and cell death in human carotid plaques: implications for plaque stabilization. Circulation. 2001; 103:926-33.

Ref ID: 107416
(42) Rattazzi M, Puato M, Faggin E, Bertipaglia B, Zambon A, Pauletto P. C-reactive protein and interleukin-6 in vascular disease: culprits or passive bystanders? J Hypertens. 2003; 21:1787-803.

Ref ID: 108597
(43) Zhang D X, Zou A P, Li P L. Ceramide-induced activation of NADPH oxidase and endothelial dysfunction in small coronary arteries. Am J Physiol Heart Circ Physiol. 2003; 284:H605-H612.

Ref ID: 108242
(44) Williams H C, Griendling K K. NADPH oxidase inhibitors: new antihypertensive agents? J Cardiovasc Pharmacol. 2007; 50:9-16.

Ref ID: 108254
(45) Yu J, Weiwer M, Linhardt R J, Dordick J S. The role of the methoxyphenol apocynin, a vascular NADPH oxidase inhibitor, as a chemopreventative agent in the potential treatment of cardiovascular diseases. Curr Vasc Pharmacol. 2008; 6:204-17.

Ref ID: 108353
(46) Lee S B, Bae I H, Bae Y S, Um H D. Link between mitochondria and NADPH oxidase 1 isozyme for the sustained production of reactive oxygen species and cell death. J Biol Chem. 2006; 281:36228-35.

Ref ID: 108240
(47) Shatwell K P, Segal A W. NADPH oxidase. Int J Biochem Cell Biol. 1996; 28:1191-95.

Ref ID: 108244
(48) Morel F, Doussiere J, Vignais P V. The superoxide-generating oxidase of phagocytic cells. Physiological, molecular and pathological aspects. Eur J Biochem. 1991; 201:523-46.

Ref ID: 108245
(49) Landmesser U, Dikalov S, Price S R, McCann L, Fukai T, Holland S M et al. Oxidation of tetrahydrobiopterin leads to uncoupling of endothelial cell nitric oxide synthase in hypertension. J Clin Invest. 2003; 111:1201-9.

Ref ID: 108566
(50) Schulz E, Jansen T, Wenzel P, Daiber A, Munzel T. Nitric oxide, tetrahydrobiopterin, oxidative stress, and endothelial dysfunction in hypertension. Antioxid Redox Signal. 2008; 10:1115-26.

Ref ID: 108564
(51) Fujii S, Zhang L, Igarashi J, Kosaka H. L-arginine reverses p47phox and gp91phox expression induced by high salt in Dahl rats. Hypertension. 2003; 42:1014-20.

Ref ID: 108586
(52) Christ M, Bauersachs J, Liebetrau C, Heck M, Gunther A, Wehling M. Glucose increases endothelial-dependent superoxide formation in coronary arteries by NAD(P)H oxidase activation: attenuation by the 3-hydroxy-3-methylglutaryl coenzyme A reductase inhibitor atorvastatin. Diabetes. 2002; 51:2648-52.

Ref ID: 108573
(53) Delbosc S, Morena M, Djouad F, Ledoucen C, Descomps B, Cristol J P. Statins, 3-hydroxy-3-methylglutaryl coenzyme A reductase inhibitors, are able to reduce superoxide anion production by NADPH oxidase in THP-1-derived monocytes. J Cardiovasc Pharmacol. 2002; 40:611-17.

Ref ID: 108574
(54) Ejiri J, Inoue N, Tsukube T, Munezane T, Hino Y, Kobayashi S et al. Oxidative stress in the pathogenesis of thoracic aortic aneurysm: protective role of statin and angiotensin II type 1 receptor blocker. Cardiovasc Res. 2003; 59:988-96.

Ref ID: 108576
(55) Nakagami H, Jensen K S, Liao J K. A novel pleiotropic effect of statins: prevention of cardiac hypertrophy by cholesterol-independent mechanisms. Ann Med. 2003; 35:398-403.

Ref ID: 108577
(56) Rueckschloss U, Galle J, Holtz J, Zerkowski H R, Morawietz H. Induction of NAD(P)H oxidase by oxidized low-density lipoprotein in human endothelial cells: antioxidative potential of hydroxymethylglutaryl coenzyme A reductase inhibitor therapy. Circulation. 2001; 104:1767-72.

Ref ID: 108570
(57) Wassmann S, Laufs U, Muller K, Konkol C, Ahlbory K, Baumer A T et al. Cellular antioxidant effects of atorvastatin in vitro and in vivo. Arterioscler Thromb Vasc Biol. 2002; 22:300-305.

Ref ID: 108571
(58) Wassmann S, Laufs U, Baumer A T, Muller K, Ahlbory K, Linz W et al. HMG-CoA reductase inhibitors improve endothelial dysfunction in normocholesterolemic hypertension via reduced production of reactive oxygen species. Hypertension. 2001; 37:1450-1457.

Ref ID: 108567
(59) Hurlimann D, Enseleit F, Ruschitzka F. [Rheumatoid arthritis, inflammation, and atherosclerosis]. Herz. 2004; 29:760-768.

Ref ID: 108598
(60) Davignon J. Beneficial cardiovascular pleiotropic effects of statins. Circulation. 2004; 109:11139-11143.

Ref ID: 108594
(61) Maron D J, Fazio S, Linton M F. Current perspectives on statins. Circulation. 2000; 101:207-13.

Ref ID: 108595
(62) Vaughan C J, Gotto A M, Jr., Basson C T. The evolving role of statins in the management of atherosclerosis. J Am Coll Cardiol. 2000; 35:1-10.

Ref ID: 108596
(63) Leitinger N. Cholesteryl ester oxidation products in atherosclerosis. Mol Aspects Med. 2003; 24:239-50.

Ref ID: 107608
(64) Sugii S, Lin S, Ohgami N, Ohashi M, Chang C C, Chang T Y. Roles of endogenously synthesized sterols in the endocytic pathway. J Biol Chem. 2006; 281:23191-206.

Ref ID: 107607
(65) Wang Y, Castoreno A B, Stockinger W, Nohturfft A. Modulation of endosomal cholesteryl ester metabolism by membrane cholesterol. J Biol Chem. 2005; 280:11876-86.

Ref ID: 107609
(66) Stenvinkel P, Ketteler M, Johnson R J, Lindholm B, Pecoits-Filho R, Riella M et al. IL-10, IL-6, and TNF-alpha: central factors in the altered cytokine network of uremia—the good, the bad, and the ugly. Kidney Int. 2005; 67:1216-33.

Ref ID: 108599
(67) Terkeltaub R, Solan J, Barry M, Jr., Santoro D, Bokoch G M. Role of the mevalonate pathway of isoprenoid synthesis in IL-8 generation by activated monocytic cells. J Leukoc Biol. 1994; 55:749-55.

Ref ID: 108617
(68) Kumar S. The apoptotic cysteine protease CPP32. Int J Biochem Cell Biol. 1997; 29:393-96.

Ref ID: 108602
(69) Thornberry N A, Bull H G, Calaycay J R, Chapman K T, Howard A D, Kostura M J et al. A novel heterodimeric cysteine protease is required for interleukin-1 beta processing in monocytes. Nature. 1992; 356:768-74.

Ref ID: 108600
(70) Thornberry N A, Molineaux S M. Interleukin-1 beta converting enzyme: a novel cysteine protease required for IL-1 beta production and implicated in programmed cell death. Protein Sci. 1995; 4:3-12.

Ref ID: 108601
(71) Jerome W G, Cox B E, Griffin E E, Ullery J C. Lysosomal cholesterol accumulation inhibits subsequent hydrolysis of lipoprotein cholesteryl ester. Microsc Microanal. 2008; 14:138-49.

Ref ID: 108195
(72) Davignon J, Leiter L A. Ongoing clinical trials of the pleiotropic effects of statins. Vasc Health Risk Manag. 2005; 1:29-40.

Ref ID: 107392
(73) Jantzen F, Koneman S, Wolff B, Barth S, Staudt A, Kroemer H K et al. Isoprenoid depletion by statins antagonizes cytokine-induced down-regulation of endothelial nitric oxide expression and increases NO synthase activity in human umbilical vein endothelial cells. J Physiol Pharmacol. 2007; 58:503-14.

Ref ID: 107535
(74) Hernandez-Perera O, Perez-Sala D, Navarro-Antolin J, Sanchez-Pascuala R, Hernandez G, Diaz C et al. Effects of the 3-hydroxy-3-methylglutaryl-CoA reductase inhibitors, atorvastatin and simvastatin, on the expression of endothelin-1 and endothelial nitric oxide synthase in vascular endothelial cells. J Clin Invest. 1998; 101:2711-19.

Ref ID: 107453
(75) Dumont Y, D'Amours M, Lebel M, Lariviere R. Supplementation with a low dose of L-arginine reduces blood pressure and endothelin-1 production in hypertensive uraemic rats. Nephrol Dial Transplant. 2001; 16:746-54.

Ref ID: 108653
(76) Phivthong-ngam L, Bode-Boger S M, Boger R H, Bohme M, Brandes R P, Mugge A et al. Dietary L-arginine normalizes endothelin-induced vascular contractions in cholesterol-fed rabbits. J Cardiovasc Pharmacol. 1998; 32:300-307.

Ref ID: 108651
(77) Mueck A O, Seeger H, Lippert T H. Fluvastatin reduces endothelin secretion of cultured human umbilical vein endothelial cells. Eur J Clin Pharmacol. 1999; 55:625-26.

Ref ID: 108656
(78) Seeger H, Mueck A O, Lippert T H. Fluvastatin increases prostacyclin and decreases endothelin production by human umbilical vein endothelial cells. Int J Clin Pharmacol Ther. 2000; 38:270-272.

Ref ID: 108657
(79) Lopez F A, Riesco A, Moliz M, Egido J, Casado S, Hernando L et al. Inhibition by L-arginine of the endothelin-mediated increase in cytosolic calcium in human neutrophils. Biochem Biophys Res Commun. 1991; 178:884-91.

Ref ID: 108650
(80) Luscher T F, Tanner F C, Noll G. Lipids and endothelial function: effects of lipid-lowering and other therapeutic interventions. Curr Opin Lipidol. 1996; 7:234-40.

Ref ID: 4447
(81) O'Meara S J, Kinsella B T. Effect of the statin atorvastatin on intracellular signalling by the prostacyclin receptor in vitro and in vivo. Br J Pharmacol. 2004; 143:292-302.

Ref ID: 108658
(82) Mehta S, Stewart D J, Langleben D, Levy R D. Short-term pulmonary vasodilation with L-arginine in pulmonary hypertension. Circulation. 1995; 92:1539-45.

Ref ID: 108660
(83) Wang Y Y, Wang J Y, Fu Y L, Wang C, Peng S Q. [Inhibition of platelet function by L-arginine.L-aspartate salt]. Sheng Li Xue Bao. 2001; 53:303-6.

Ref ID: 108659
(84) Bode-Boger S M, Boger R H, Kienke S, Bohme M, Phivthong-ngam L, Tsikas D et al. Chronic dietary supplementation with L-arginine inhibits platelet aggregation and thromboxane A2 synthesis in hypercholesterolaemic rabbits in vivo. Cardiovasc Res. 1998; 37:756-64.

Ref ID: 5
(85) Laufs U, Liao J K, Bohm M. Lipid management with statins. The lower the better? Z Kardiol. 2004; 93:4-9.

Ref ID: 107586
(86) Davignon J. Beneficial cardiovascular pleiotropic effects of statins. Circulation. 2004; 109:11139-11143.

Ref ID: 107389
(87) Jukema J W, Bruschke A V, van Boven A J, Reiber J H, Bal E T, Zwinderman A H et al. Effects of lipid lowering by pravastatin on progression and regression of coronary artery disease in symptomatic men with normal to moderately elevated serum cholesterol levels. The Regression Growth Evaluation Statin Study (REGRESS). Circulation. 1995; 91:2528-40.

Ref ID: 107395
(88) Sacks F M, Gibson C M, Rosner B, Pasternak R C, Stone P H. The influence of pretreatment low density lipoprotein cholesterol concentrations on the effect of hypocholesterolemic therapy on coronary atherosclerosis in angiographic trials. Harvard Atherosclerosis Reversibility Project Research Group. Am J Cardiol. 1995; 76:78C-85C.

Ref ID: 107400
(89) Study Group. Baseline serum cholesterol and treatment effect in the Scandinavian Simvastatin Survival Study (4S). Lancet. 1995; 345:1274-75.

Ref ID: 107403
(90) Study Group. Influence of pravastatin and plasma lipids on clinical events in the West of Scotland Coronary Prevention Study (WOSCOPS). Circulation. 1998; 97:1440-1445.

Ref ID: 107404
(91) Thompson G R, Hollyer J, Waters D D. Percentage change rather than plasma level of LDL-cholesterol determines therapeutic response in coronary heart disease. Curr Opin Lipidol. 1995; 6:386-88.

Ref ID: 107405
(92) Marz W, Scharnagl H, Abletshauser C, Hoffmann M M, Berg A, Keul J et al. Fluvastatin lowers atherogenic dense low-density lipoproteins in postmenopausal women with the atherogenic lipoprotein phenotype. Circulation. 2001; 103:1942-48.

Ref ID: 107512
(93) Otvos J D, Shalaurova I, Freedman D S, Rosenson R S. Effects of pravastatin treatment on lipoprotein subclass profiles and particle size in the PLAC-I trial. Atherosclerosis. 2002; 160:41-48.

Ref ID: 107514
(94) Nissen S E, Nicholls S J, Sipahi I, Libby P, Raichlen J S, Ballantyne C M et al. Effect of very high-intensity statin therapy on regression of coronary atherosclerosis: the ASTEROID trial. JAMA. 2006; 295:1556-65.

Ref ID: 107398
(95) Murphy S A, Cannon C P, Wiviott S D, de Lemos J A, Blazing M A, McCabe C H et al. Effect of intensive lipid-lowering therapy on mortality after acute coronary syndrome (a patient-level analysis of the Aggrastat to Zocor and Pravastatin or Atorvastatin Evaluation and Infection Therapy-Thrombolysis in Myocardial Infarction 22 trials). Am J Cardiol. 2007; 100:1047-51.

Ref ID: 107408
(96) Scirica B M, Morrow D A, Cannon C P, Ray K K, Sabatine M S, Jarolim P et al. Intensive statin therapy and the risk of hospitalization for heart failure after an acute coronary syndrome in the PROVE IT-TIMI 22 study. J Am Coll Cardiol. 2006; 47:2326-31.

Ref ID: 107407
(97) Ambrose J A, Martinez E E. A new paradigm for plaque stabilization. Circulation. 2002; 105:2000-2004.

Ref ID: 107417
(98) Ridker P M, Rifai N, Pfeffer M A, Sacks F, Braunwald E. Long-term effects of pravastatin on plasma concentration of C-reactive protein. The Cholesterol and Recurrent Events (CARE) Investigators. Circulation. 1999; 100:230-235.

Ref ID: 107427
(99) Jialal I, Stein D, Balis D, Grundy S M, ms-Huet B, Devaraj S. Effect of hydroxymethyl glutaryl coenzyme a reductase inhibitor therapy on high sensitive C-reactive protein levels. Circulation. 2001; 103:1933-35.

Ref ID: 107424
(100) Dangas G, Smith D A, Unger A H, Shao J H, Meraj P, Fier C et al. Pravastatin: an antithrombotic effect independent of the cholesterol-lowering effect. Thromb Haemost. 2000; 83:688-92.

Ref ID: 107435
(101) Martinez-Gonzalez J, Badimon L. Influence of statin use on endothelial function: from bench to clinics. Curr Pharm Des. 2007; 13:1771-86.

Ref ID: 107527
(102) Tawfik H E, El-Remessy A B, Matragoon S, Ma G, Caldwell R B, Caldwell R W. Simvastatin improves diabetes-induced coronary endothelial dysfunction. J Pharmacol Exp Ther. 2006; 319:386-95.

Ref ID: 107546
(103) Tousoulis D, Antoniades C, Koumallos N, Marinou K, Stefanadi E, Latsios G et al. Novel therapies targeting vascular endothelium. Endothelium. 2006; 13:411-21.

Ref ID: 107542
(104) Kaesemeyer W H, Caldwell R B, Huang J, Caldwell R W. Pravastatin sodium activates endothelial nitric oxide synthase independent of its cholesterol-lowering actions. J Am Coll Cardiol. 1999; 33:234-41.

Ref ID: 107454
(105) Kawakami A, Tanaka A, Nakajima K, Shimokado K, Yoshida M. Atorvastatin attenuates remnant lipoprotein-induced monocyte adhesion to vascular endothelium under flow conditions. Circ Res. 2002; 91:263-71.

Ref ID: 107566
(106) Serrano C V, Jr., Yoshida V M, Venturinelli M L, D'Amico E, Monteiro H P, Ramires J A et al. Effect of simvastatin on monocyte adhesion molecule expression in patients with hypercholesterolemia. Atherosclerosis. 2001; 157:505-12.

Ref ID: 107568
(107) Portal V L, Moriguchi E H, Vieira J L, Schio S, Mastalir E T, Buffe F et al. Comparison of the effect of two HMG CoA reductase inhibitors on LDL susceptibility to oxidation. Arq Bras Cardiol. 2003; 80:156-5.

Ref ID: 107569
(108) Chiu J H, Abdelhadi R H, Chung M K, Gurm H S, Marrouche N F, Saliba W I et al. Effect of statin therapy on risk of ventricular arrhythmia among patients with coronary artery disease and an implantable cardioverter-defibrillator. Am J Cardiol. 2005; 95:490-491.

Ref ID: 107570
(109) van Boven A J, Jukema J W, Zwinderman A H, Crijns H J, Lie K I, Bruschke A V. Reduction of transient myocardial ischemia with pravastatin in addition to the conventional treatment in patients with angina pectoris. REGRESS Study Group. Circulation. 1996; 94:1503-5.

Ref ID: 107462
(110) Eichstadt H W, Eskotter H, Hoffman I, Amthauer H W, Weidinger G. Improvement of myocardial perfusion by short-term fluvastatin therapy in coronary artery disease. Am J Cardiol. 1995; 76:122A-5A.

Ref ID: 107465
(111) Freeman D J, Norrie J, Caslake M J, Gaw A, Ford I, Lowe G D et al. C-reactive protein is an independent predictor of risk for the development of diabetes in the West of Scotland Coronary Prevention Study. Diabetes. 2002; 51:1596-600.

Ref ID: 107494
(112) Freeman D J, Norrie J, Sattar N, Neely R D, Cobbe S M, Ford I et al. Pravastatin and the development of diabetes mellitus: evidence for a protective treatment effect in the West of Scotland Coronary Prevention Study. Circulation. 2001; 103:357-62.

Ref ID: 107493
(113) Sposito A C, Mansur A P, Coelho O R, Nicolau J C, Ramires J A. Additional reduction in blood pressure after cholesterol-lowering treatment by statins (lovastatin or pravastatin) in hypercholesterolemic patients using angiotensin-converting enzyme inhibitors (enalapril or lisinopril). Am J Cardiol. 1999; 83:1497-9, A8.

Ref ID: 107499
(114) Tonolo G, Melis M G, Foimato M, Angius M F, Carboni A, Brizzi P et al. Additive effects of Simvastatin beyond its effects on LDL cholesterol in hypertensive type 2 diabetic patients. Eur J Clin Invest. 2000; 30:980-987.

Ref ID: 107500
(115) Horwich T B, MacLellan W R, Fonarow G C. Statin therapy is associated with improved survival in ischemic and non-ischemic heart failure. J Am Coll Cardiol. 2004; 43:642-48.

Ref ID: 107501
(116) Mozaffarian D, Nye R, Levy W C. Statin therapy is associated with lower mortality among patients with severe heart failure. Am J Cardiol. 2004; 93:1124-29.

Ref ID: 107502
(117) Chu C S, Kou H S, Lee C J, Lee K T, Chen S H, Voon W C et al. Effect of atorvastatin withdrawal on circulating coenzyme Q10 concentration in patients with hypercholesterolemia. Biofactors. 2006; 28:177-84.

Ref ID: 107384
(118) Mortensen S A, Leth A, Agner E, Rohde M. Dose-related decrease of serum coenzyme Q10 during treatment with HMG-CoA reductase inhibitors. Mol Aspects Med. 1997; 18 Suppl:S137-S144.

Ref ID: 107375
(119) De P G, Chariot P, mmi-Said M, Louarn F, Lejonc J L, Astier A et al. Lipid-lowering drugs and mitochondrial function: effects of HMG-CoA reductase inhibitors on serum ubiquinone and blood lactate/pyruvate ratio. Br J Clin Pharmacol. 1996; 42:333-37.

Ref ID: 107369
(120) Ghirlanda G, Oradei A, Manto A, Lippa S, Uccioli L, Caputo S et al. Evidence of plasma CoQ10-lowering effect by HMG-CoA reductase inhibitors: a double-blind, placebo-controlled study. J Clin Pharmacol. 1993; 33:226-29.

Ref ID: 107479
(121) Phillips P S, Phillips C T, Sullivan M J, Naviaux R K, Haas R H. Statin myotoxicity is associated with changes in the cardiopulmonary function. Atherosclerosis. 2004; 177:183-88.

Ref ID: 107488
(122) Ballantyne C M, Corsini A, Davidson M H, Holdaas H, Jacobson T A, Leitersdorf E et al. Risk for myopathy with statin therapy in high-risk patients. Arch Intern Med. 2003; 163:553-64.

Ref ID: 107491
(123) Thompson P D, Clarkson P, Karas R H. Statin-associated myopathy. JAMA. 2003; 289:1681-90.

Ref ID: 107484
(124) Phillips P S, Haas R H, Bannykh S, Hathaway S, Gray N L, Kimura B J et al. Statin-associated myopathy with normal creatine kinase levels. Ann Intern Med. 2002; 137:581-85.

Ref ID: 107487
(125) Folkers K, Langsjoen P, Willis R, Richardson P, Xia L J, Ye C Q et al. Lovastatin decreases coenzyme Q levels in humans. Proc Natl Acad Sci USA. 1990; 87:8931-34.

Ref ID: 98686
(126) Bhardwaj S S, Chalasani N. Lipid-lowering agents that cause drug-induced hepatotoxicity. Clin Liver Dis. 2007; 11:597-613, vii.

Ref ID: 107575
(127) Clarke A T, Mills P R. Atorvastatin associated liver disease. Dig Liver Dis. 2006; 38:772-77.

Ref ID: 107573
(128) Khorashadi S, Hasson N K, Cheung R C. Incidence of statin hepatotoxicity in patients with hepatitis C. Clin Gastroenterol Hepatol. 2006; 4:902-7.

Ref ID: 107579
(129) Lewis J H, Mortensen M E, Zweig S, Fusco M J, Medoff J R, Belder R. Efficacy and safety of high-dose pravastatin in hypercholesterolemic patients with well-compensated chronic liver disease: Results of a prospective, randomized, double-blind, placebo-controlled, multicenter trial. Hepatology. 2007; 46:1453-63.

Ref ID: 107576
(130) Tavintharan S, Ong C N, Jeyaseelan K, Sivakumar M, Lim S C, Sum C F. Reduced mitochondrial coenzyme Q10 levels in HepG2 cells treated with high-dose simvastatin: a possible role in statin-induced hepatotoxicity? Toxicol Appl Pharmacol. 2007; 223:173-79.

Ref ID: 107577
(131) Igel M, Sudhop T, von B K. [Pleiotrophic effect of statins (3-hydroxy-3-methylglutaryl-coenzyme a reductase inhibitors)]. Arzneimittelforschung. 2003; 53:545-53.

Ref ID: 107612
(132) Maron D J, Fazio S, Linton M F. Current perspectives on statins. Circulation. 2000; 101:207-13.

Ref ID: 107397
(133) Ballantyne C M, Blazing M A, Hunninghake D B, Davidson M H, Yuan Z, DeLucca P et al. Effect on high-density lipoprotein cholesterol of maximum dose simvastatin and atorvastatin in patients with hypercholesterolemia: results of the Comparative HDL Efficacy and Safety Study (CHESS). Am Heart J. 2003; 146:862-69.

Ref ID: 107492
(134) Corti R, Fayad Z A, Fuster V, Worthley S G, Helft G, Chesebro J et al. Effects of lipid-lowering by simvastatin on human atherosclerotic lesions: a longitudinal study by high-resolution, noninvasive magnetic resonance imaging. Circulation. 2001; 104:249-52.

Ref ID: 107411
(135) Brown B G, Zhao X Q, Chait A, Fisher L D, Cheung M C, Morse J S et al. Simvastatin and niacin, antioxidant vitamins, or the combination for the prevention of coronary disease. N Engl J Med. 2001; 345:1583-92.

Ref ID: 107413
(136) Study Group. Effect of simvastatin on coronary atheroma: the Multicentre Anti-Atheroma Study (MAAS). Lancet. 1994; 344:633-38.

Ref ID: 107412
(137) Schartl M, Bocksch W, Koschyk D H, Voelker W, Karsch K R, Kreuzer J et al. Use of intravascular ultrasound to compare effects of different strategies of lipid-lowering therapy on plaque volume and composition in patients with coronary artery disease. Circulation. 2001; 104:387-92.

Ref ID: 107414
(138) Williams J K, Sukhova G K, Herrington D M, Libby P. Pravastatin has cholesterol-lowering independent effects on the artery wall of atherosclerotic monkeys. J Am Coll Cardiol. 1998; 31:684-91.

Ref ID: 107415
(139) Schartl M, Bocksch W, Fateh-Moghadam S. Effects of lipid-lowering therapy on coronary artery remodeling. Coron Artery Dis. 2004; 15:45-51.

Ref ID: 107418
(140) Falk E, Shah P K, Fuster V. Coronary plaque disruption. Circulation. 1995; 92:657-71.

Ref ID: 107420
(141) Libby P. Molecular bases of the acute coronary syndromes. Circulation. 1995; 91:2844-50.

Ref ID: 107419
(142) Aikawa M, Rabkin E, Sugiyama S, Voglic S J, Fukumoto Y, Furukawa Y et al. An HMG-CoA reductase inhibitor, cerivastatin, suppresses growth of macrophages expressing matrix metalloproteinases and tissue factor in vivo and in vitro. Circulation. 2001; 103:276-83.

Ref ID: 107421
(143) Bellosta S, Via D, Canavesi M, Pfister P, Fumagalli R, Paoletti R et al. HMG-CoA reductase inhibitors reduce MMP-9 secretion by macrophages. Arterioscler Thromb Vasc Biol. 1998; 18:1671-78.

Ref ID: 107422
(144) Albert M A, Danielson E, Rifai N, Ridker P M. Effect of statin therapy on C-reactive protein levels: the pravastatin inflammation/CRP evaluation (PRINCE): a randomized trial and cohort study. JAMA. 2001; 286:64-70.

Ref ID: 108680
(145) Jialal I, Stein D, Balis D, Grundy S M, ms-Huet B, Devaraj S. Effect of hydroxymethyl glutaryl coenzyme a reductase inhibitor therapy on high sensitive C-reactive protein levels. Circulation. 2001; 103:1933-35.

Ref ID: 108677
(146) Plenge J K, Hernandez T L, Weil K M, Poirier P, Grunwald G K, Marcovina S M et al. Simvastatin lowers C-reactive protein within 14 days: an effect independent of low-density lipoprotein cholesterol reduction. Circulation. 2002; 106:1447-52.

Ref ID: 108676
(147) Ridker P M, Rifai N, Pfeffer M A, Sacks F, Braunwald E. Long-term effects of pravastatin on plasma concentration of C-reactive protein. The Cholesterol and Recurrent Events (CARE) Investigators. Circulation. 1999; 100:230-235.

Ref ID: 108679
(148) Ridker P M, Danielson E, Fonseca F A, Genest J, Gotto A M, Jr., Kastelein J J et al. Rosuvastatin to Prevent Vascular Events in Men and Women with Elevated C-Reactive Protein. N Engl J Med. 2008.

Ref ID: 108669
(149) Rosenson R S, Tangney C C, Levine D M, Parker T S, Gordon B R. Association between reduced low density lipoprotein oxidation and inhibition of monocyte chemoattractant protein-1 production in statin-treated subjects. J Lab Clin Med. 2005; 145:83-87.

Ref ID: 107588
(150) Undas A, Brummel K E, Musial J, Mann K G, Szczeklik A. Simvastatin depresses blood clotting by inhibiting activation of prothrombin, factor V, and factor XIII and by enhancing factor Va inactivation. Circulation. 2001; 103:2248-53.

Ref ID: 107592
(151) Dangas G, Badimon J J, Smith D A, Unger A H, Levine D, Shao J H et al. Pravastatin therapy in hyperlipidemia: effects on thrombus formation and the systemic hemostatic profile. J Am Coll Cardiol. 1999; 33:1294-304.

Ref ID: 107468
(152) Lacoste L, Lam J Y, Hung J, Letchacovski G, Solymoss C B, Waters D. Hyperlipidemia and coronary disease. Correction of the increased thrombogenic potential with cholesterol reduction. Circulation. 1995; 92:3172-77.

Ref ID: 107437
(153) Mayer J, Eller T, Brauer P, Solleder E M, Schafer R M, Keller F et al. Effects of long-term treatment with lovastatin on the clotting system and blood platelets. Ann Hematol. 1992; 64:196-201.

Ref ID: 107439
(154) Lindmark E, Tenno T, Siegbahn A. Role of platelet P-selectin and CD40 ligand in the induction of monocytic tissue factor expression. Arterioscler Thromb Vasc Biol. 2000; 20:2322-28.

Ref ID: 107438
(155) Aukrust P, Muller F, Ueland T, Berget T, Aaser E, Brunsvig A et al. Enhanced levels of soluble and membrane-bound CD40 ligand in patients with unstable angina. Possible reflection of T lymphocyte and platelet involvement in the pathogenesis of acute coronary syndromes. Circulation. 1999; 100:614-20.

Ref ID: 107440
(156) Schonbeck U, Varo N, Libby P, Buring J, Ridker P M. Soluble CD40L and cardiovascular risk in women. Circulation. 2001; 104:2266-68.

Ref ID: 107441
(157) Behrendt D, Ganz P. Endothelial function. From vascular biology to clinical applications. Am J Cardiol. 2002; 90:40L-8L.

Ref ID: 107614
(158) Egashira K, Hirooka Y, Kai H, Sugimachi M, Suzuki S, Inou T et al. Reduction in serum cholesterol with pravastatin improves endothelium-dependent coronary vasomotion in patients with hypercholesterolemia. Circulation. 1994; 89:2519-24.

Ref ID: 107446
(159) Treasure C B, Klein J L, Weintraub W S, Talley J D, Stillabower M E, Kosinski A S et al. Beneficial effects of cholesterol-lowering therapy on the coronary endothelium in patients with coronary artery disease. N Engl J Med. 1995; 332:481-87.

Ref ID: 107449
(160) Tsunekawa T, Hayashi T, Kano H, Sumi D, Matsui-Hirai H, Thakur N K et al. Cerivastatin, a hydroxymethylglutaryl coenzyme a reductase inhibitor, improves endothelial function in elderly diabetic patients within 3 days. Circulation. 2001; 104:376-79.

Ref ID: 107450
(161) Myers P R, Banitt P F, Guerra R, Jr., Harrison D G. Role of the endothelium in modulation of the acetylcholine vasoconstrictor response in porcine coronary microvessels. Cardiovasc Res. 1991; 25:129-37.

Ref ID: 4572
(162) Busse R, Hecker M, Fleming I. Control of nitric oxide and prostacyclin synthesis in endothelial cells. Arzneimittelforschung. 1994; 44:392-6.

Ref ID: 3782
(163) Otsuji S, Nakajima O, Waku S, Kojima S, Hosokawa H, Kinoshita I et al. Attenuation of acetylcholine-induced vasoconstriction by L-arginine is related to the progression of atherosclerosis. Am Heart J. 1995; 129:1094-100.

Ref ID: 4654
(164) Arnal J F. [Nitric oxide and circulatory homeostasis]. Ann Cardiol Angeiol (Paris). 1997; 46:420-5.

Ref ID: 3648
(165) Dupuis J, Tardif J C, Rouleau J L, Ricci J, Arnold M, Lonn E et al. Intensity of lipid lowering with statins and brachial artery vascular endothelium reactivity after acute coronary syndromes (from the BRAVER trial). Am J Cardiol. 2005; 96:1207-13.

Ref ID: 107445
(166) Lefer A M, Campbell B, Shin Y K, Scalia R, Hayward R, Lefer D J. Simvastatin preserves the ischemic-reperfused myocardium in normocholesterolemic rat hearts. Circulation. 1999; 100:178-84.

Ref ID: 107448
(167) Vita J A, Yeung A C, Winniford M, Hodgson J M, Treasure C B, Klein J L et al. Effect of cholesterol-lowering therapy on coronary endothelial vasomotor function in patients with coronary artery disease. Circulation. 2000; 102:846-51.

Ref ID: 107451
(168) Yokoyama I, Momomura S, Ohtake T, Yonekura K, Yang W, Kobayakawa N et al. Improvement of impaired myocardial vasodilatation due to diffuse coronary atherosclerosis in hypercholesterolemics after lipid-lowering therapy. Circulation. 1999; 100:117-22.

Ref ID: 107452
(169) John S, Schneider M P, Delles C, Jacobi J, Schmieder R E. Lipid-independent effects of statins on endothelial function and bioavailability of nitric oxide in hypercholesterolemic patients. Am Heart J. 2005; 149:473.

Ref ID: 107553
(170) Kalinowski L, Dobrucki L W, Brovkovych V, Malinski T. Increased nitric oxide bioavailability in endothelial cells contributes to the pleiotropic effect of cerivastatin. Circulation. 2002; 105:933-38.

Ref ID: 107447
(171) Laufs U, La Fata V, Plutzky J, Liao J K. Upregulation of endothelial nitric oxide synthase by HMG CoA reductase inhibitors. Circulation. 1998; 97:1129-35.

Ref ID: 6621
(172) Mitchell L B, Powell J L, Gillis A M, Kehl V, Hallstrom A P. Are lipid-lowering drugs also antiarrhythmic drugs? An analysis of the Antiarrhythmics versus Implantable Defibrillators (AVID) trial. J Am Coll Cardiol. 2003; 42:81-87.

Ref ID: 107596
(173) Ajioka M, Nagai S, Ogawa K, Satake T, Sugiyama S, Ozawa T. The role of phospholipase in the genesis of reperfusion arrhythmia. J Electrocardiol. 1986; 19:165-72.

Ref ID: 108763
(174) Imai S, Matsui K, Nakazawa M, Takatsuka N, Takeda K, Tamatsu H. Anti-arrhythmic effects of (−)-carnitine chloride and its acetyl analogue on canine late ventricular arrhythmia induced by ligation of the coronary artery as related to improvement of mitochondrial function. Br J Pharmacol. 1984; 82:533-42.

Ref ID: 108764
(175) Kitazawa M, Sugiyama S, Ozawa T, Miyazaki Y, Kotaka K. Mechanism of chlorpromazine-induced arrhythmia—arrhythmia and mitochondrial dysfunction. J Electrocardiol. 1981; 14:219-24.

Ref ID: 108765
(176) Martina B, Zuber M, Weiss P, Burkart F, Ritz R. [Anti-arrhythmia treatment using L-carnitine in acute myocardial infarct]. Schweiz Med Wochenschr. 1992; 122:1352-55.

Ref ID: 108761
(177) Mondillo S, Faglia S, D'Aprile N, Mangiacotti L, Campolo M A, Agricola E et al. [Therapy of arrhythmia induced by myocardial ischemia. Association of L-carnitine, propafenone and mexiletine]. Clin Ter. 1995; 146:769-74.

Ref ID: 108760
(178) Wang Y L, Li Y S, Fu S X. Effect of ubiquinone on ischemic arrhythmia in conscious rats. Zhongguo Yao Li Xue Bao. 1991; 12:202-6.

Ref ID: 108762
(179) Shen J, Wang J, Zhao B, Hou J, Gao T, Xin W. Effects of EGb 761 on nitric oxide and oxygen free radicals, myocardial damage and arrhythmia in ischemia-reperfusion injury in vivo. Biochim Biophys Acta. 1998; 1406:228-36.

Ref ID: 108759
(180) Saltzman E A, Guay A T, Jacobson J. Improvement in erectile function in men with organic erectile dysfunction by correction of elevated cholesterol levels: a clinical observation. J Urol. 2004; 172:255-58.

Ref ID: 107531
(181) Miner M, Billups K L. Erectile dysfunction and dyslipidemia: relevance and role of phosphodiesterase type-5 inhibitors and statins. J Sex Med. 2008; 5:1066-78.

Ref ID: 108668
(182) Kaufman S. Tetrahydrobiopterin. 1 ed. 1997.

Ref ID: 105997
(183) Cosentino F, Luscher T F. Tetrahydrobiopterin and endothelial function. Eur Heart J. 1998; 19 Suppl G:G3-G8.

Ref ID: 82674
(184) Maier W, Cosentino F, Lutolf R B, Fleisch M, Seiler C, Hess O M et al. Tetrahydrobiopterin improves endothelial function in patients with coronary artery disease. J Cardiovasc Pharmacol. 2000; 35:173-78.

Ref ID: 16213
(185) Sommer F, Klotz T, Steinritz D, Bloch W. Evaluation of tetrahydrobiopterin (BH4) as a potential therapeutic agent to treat erectile dysfunction. Asian J Androl. 2006; 8:159-67.

Ref ID: 106604
(186) Wang B Y, Ho H K, Lin P S, Schwarzacher S P, Pollman M J, Gibbons G H et al. Regression of atherosclerosis: role of nitric oxide and apoptosis. Circulation. 1999; 99:1236-41.

Ref ID: 11873
(187) Bode-Boger S M, Boger R H, Kienke S, Bohme M, Phivthong-ngam L, Tsikas D et al. Chronic dietary supplementation with L-arginine inhibits platelet aggregation and thromboxane A2 synthesis in hypercholesterolaemic rabbits in vivo. Cardiovasc Res. 1998; 37:756-64.

Ref ID: 108661
(188) Adams M R, Jessup W, Celermajer D S. Cigarette smoking is associated with increased human monocyte adhesion to endothelial cells: reversibility with oral L-arginine but not vitamin C. J Am Coll Cardiol. 1997; 29:491-97.

Ref ID: 72094
(189) Adams M R, Jessup W, Hailstones D, Celermajer D S. L-arginine reduces human monocyte adhesion to vascular endothelium and endothelial expression of cell adhesion molecules [see comments]. Circulation. 1997; 95:662-8.

Ref ID: 3600
(190) Adams M R, McCredie R, Jessup W, Robinson J, Sullivan D, Celermajer D S. Oral L-arginine improves endothelium-dependent dilatation and reduces monocyte adhesion to endothelial cells in young men with coronary artery disease. Atherosclerosis. 1997; 129:261-9.

Ref ID: 3601
(191) Herman A G, Moncada S. Therapeutic potential of nitric oxide donors in the prevention and treatment of atherosclerosis. Eur Heart J. 2005; 26:1945-55.

Ref ID: 107552
(192) Maas R. Pharmacotherapies and their influence on asymmetric dimethylargine (ADMA). Vasc Med. 2005; 10 Suppl 1:S49-S57.

Ref ID: 108775
(193) Boger G I, Rudolph T K, Maas R, Schwedhelm E, Dumbadze E, Bierend A et al. Asymmetric dimethylarginine determines the improvement of endothelium-dependent vasodilation by simvastatin: Effect of combination with oral L-arginine. J Am Coll Cardiol. 2007; 49:2274-82.

Ref ID: 108772
(194) Panichi V, Mantuano E, Paoletti S, Santi S, Manca R G, Cutrupi S et al. Effect of simvastatin on plasma asymmetric dimethylarginine concentration in patients with chronic kidney disease. J Nephrol. 2008; 21:38-44.

Ref ID: 108770
(195) Pereira E C, Bertolami M C, Faludi A A, Salem M, Bersch D, Abdalla D S. Effects of simvastatin and L-arginine on vasodilation, nitric oxide metabolites and endogenous NOS inhibitors in hypercholesterolemic subjects. Free Radic Res. 2003; 37:529-36.

Ref ID: 108636
(196) Vladimirova-Kitova LG. Asymmetric dimethylarginine—mechanisms and targets for therapeutic management. Folia Med (Plovdiv). 2008; 50:12-21.

Ref ID: 108637
(197) Young J M, Strey C H, George P M, Florkowski C M, Sies C W, Frampton C M et al. Effect of atorvastatin on plasma levels of asymmetric dimethylarginine in patients with non-ischaemic heart failure. Eur J Heart Fail. 2008; 10:463-66.

Ref ID: 108638
(198) Sasaki S, Kuwahara N, Kunitomo K, Harada S, Yamada T, Azuma A et al. Effects of atorvastatin on oxidized low-density lipoprotein, low-density lipoprotein subfraction distribution, and remnant lipoprotein in patients with mixed hyperlipoproteinemia. Am J Cardiol. 2002; 89:386-89.

Ref ID: 108782
(199) Oguz A, Uzunlulu M. Short term fluvastatin treatment lowers serum asymmetric dimethylarginine levels in patients with metabolic syndrome. Int Heart J. 2008; 49:303-11.

Ref ID: 108767
(200) Lu T M, Ding Y A, Leu H B, Yin W H, Sheu W H, Chu K M. Effect of rosuvastatin on plasma levels of asymmetric dimethylarginine in patients with hypercholesterolemia. Am J Cardiol. 2004; 94:157-61.

Ref ID: 108777
(201) Tanaka N, Katayama Y, Katsumata T, Otori T, Nishiyama Y. Effects of long-term administration of HMG-CoA reductase inhibitor, atorvastatin, on stroke events and local cerebral blood flow in stroke-prone spontaneously hypertensive rats. Brain Res. 2007; 1169:125-32.

Ref ID: 108771
(202) Chen X, Niroomand F, Liu Z, Zankl A, Katus H A, Jahn L et al. Expression of nitric oxide related enzymes in coronary heart disease. Basic Res Cardiol. 2006; 101:346-53.

Ref ID: 108786
(203) Boger G I, Rudolph T K, Maas R, Schwedhelm E, Dumbadze E, Bierend A et al. Asymmetric dimethylarginine determines the improvement of endothelium-dependent vasodilation by simvastatin: Effect of combination with oral L-arginine. J Am Coll Cardiol. 2007; 49:2274-82.

Ref ID: 107538
(204) Cardounel A J, Xia Y, Zweier J L. Endogenous methylarginines modulate superoxide as well as nitric oxide generation from neuronal nitric-oxide synthase: differences in the effects of monomethyl- and dimethylarginines in the presence and absence of tetrahydrobiopterin. J Biol Chem. 2005; 280:7540-7549.

Ref ID: 108789
(205) Kurowska E M. Nitric oxide therapies in vascular diseases. Curr Pharm Des. 2002; 8:155-66.

Ref ID: 108790
(206) Gur S, Kadowitz P J, Trost L, Hellstrom W J. Optimizing nitric oxide production by time dependent L-arginine administration in isolated human corpus cavernosum. J Urol. 2007; 178:1543-48.

Ref ID: 107301
(207) Wierzbicki A S, Solomon H, Lumb P J, Lyttle K, Lambert-Hammill M, Jackson G. Asymmetric dimethyl arginine levels correlate with cardiovascular risk factors in patients with erectile dysfunction. Atherosclerosis. 2006; 185:421-25.

Ref ID: 107551
(208) Bivalacqua T J, Burnett A L, Hellstrom W J, Champion H C. Overexpression of arginase in the aged mouse penis impairs erectile function and decreases eNOS activity: influence of in vivo gene therapy of anti-arginase. Am J Physiol Heart Circ Physiol. 2007; 292:H1340-H1351.

Ref ID: 107324
(209) Vladutiu G D, Simmons Z, Isackson P J, Tarnopolsky M, Peltier W L, Barboi A C et al. Genetic risk factors associated with lipid-lowering drug-induced myopathies. Muscle Nerve. 2006; 34:153-62.

Ref ID: 107511
(210) Arduini A, Peschechera A, Giannessi F, Carminati P. Improvement of statin-associated myotoxicity by L-carnitine. J Thromb Haemost. 2004; 2:2270-2271.

Ref ID: 107508
(211) Sirtori C R, Calabresi L, Ferrara S, Pazzucconi F, Bondioli A, Baldassarre D et al. L-carnitine reduces plasma lipoprotein(a) levels in patients with hyper Lp(a). Nutr Metab Cardiovasc Dis. 2000; 10:247-51.

Ref ID: 108471
(212) Singh R B, Niaz M A, Agarwal P, Beegum R, Rastogi S S, Sachan D S. A randomised, double-blind, placebo-controlled trial of L-carnitine in suspected acute myocardial infarction. Postgrad Med J. 1996; 72:45-50.

Ref ID: 28115
(213) Shankar S S, Mirzamohammadi B, Walsh J P, Steinberg H O. L-carnitine may attenuate free fatty acid-induced endothelial dysfunction. Ann NY Acad Sci. 2004; 1033:189-97.

Ref ID: 108666
(214) Gentile V, Vicini P, Prigiotti G, Koverech A, Di S F. Preliminary observations on the use of propionyl-L-carnitine in combination with sildenafil in patients with erectile dysfunction and diabetes. Curr Med Res Opin. 2004; 20:1377-84.

Ref ID: 107332
(215) Tekle M, Bentinger M, Nordman T, Appelkvist E L, Chojnacki T, Olsson J M. Ubiquinone biosynthesis in rat liver peroxisomes. Biochem Biophys Res Commun. 2002; 291:1128-33.

Ref ID: 108710
(216) Chan T S, O'Brien P J. Hepatocyte metabolism of coenzyme Q1 (ubiquinone-5) to its sulfate conjugate decreases its antioxidant activity. Biofactors. 2003; 18:207-18.

Ref ID: 108711
(217) Singh R B, Niaz M A, Rastogi V, Rastogi S S. Coenzyme Q in cardiovascular disease. J Assoc Physicians India. 1998; 46:299-306.

Ref ID: 107379
(218) Alleva R, Tomasetti M, Battino M, Curatola G, Littarru G P, Folkers K. The roles of coenzyme Q10 and vitamin E on the peroxidation of human low density lipoprotein subfractions. Proc Natl Acad Sci USA. 1995; 92:9388-91.

Ref ID: 108725
(219) Ernster L, Dallner G. Biochemical, physiological and medical aspects of ubiquinone function. Biochim Biophys Acta. 1995; 1271:195-204.

Ref ID: 108704
(220) Zakova P, Kand'ar R, Skarydova L, Skalicky J, Myjavec A, Vojtisek P. Ubiquinol-10/lipids ratios in consecutive patients with different angiographic findings. Clin Chim Acta. 2007; 380:133-38.

Ref ID: 108702
(221) Vijan S, Hayward R A. Pharmacologic lipid-lowering therapy in type 2 diabetes mellitus: background paper for the American College of Physicians. Ann Intern Med. 2004; 140:650-658.

Ref ID: 108724
(222) Liao J K, Shin W S, Lee W Y, Clark S L. Oxidized low-density lipoprotein decreases the expression of endothelial nitric oxide synthase. J Biol Chem. 1995; 270:319-24.

Ref ID: 6671
(223) Okamoto T, Matsuya T, Fukunaga Y, Kishi T, Yamagami T. Human serum ubiquinol-10 levels and relationship to serum lipids. Int J Vitam Nutr Res. 1989; 59:288-92.

Ref ID: 107473
(224) Siegel G J. Basic neurochemistry: molecular, cellular, and medical aspects. 6th ed. Philadelphia: Lippincott-Raven Publishers; 1998.

Ref ID: 14555
(225) Molyneux S L, Florkowski C M, George P M, Piibrow A P, Frampton C M, Lever M et al. Coenzyme Q10: an independent predictor of mortality in chronic heart failure. J Am Coll Cardiol. 2008; 52:1435-41.

Ref ID: 108733
(226) Langsjoen P H, Langsjoen A M. Supplemental ubiquinol in patients with advanced congestive heart failure. Biofactors. 2008; 32:119-28.

Ref ID: 108732
(227) Gullestad L, Oie E, Ueland T, Yndestad A, Aukrust P. The role of statins in heart failure. Fundam Clin Pharmacol. 2007; 21 Suppl 2:35-40.

Ref ID: 108735
(228) Fuchs J, Packer L, Zimmer G. Lipoic Acid in Health and Disease. New York: Marcel Dekker, Inc; 1997.

Ref ID: 73116
(229) Editor. Monograph:Alpha-Lipoic Acid. Altern Med Rev. 1998; 3:308-11.

Ref ID: 8673
(230) Bierhaus A, Chevion S, Chevion M, Hofmann M, Quehenberger P, Illmer T et al. Advanced glycation end product-induced activation of NF-kappaB is suppressed by alpha-lipoic acid in cultured endothelial cells. Diabetes. 1997; 46:1481-90.

Ref ID: 5402
(231) Hofmann M A, Schiekofer S, Isermann B, Kanitz M, Henkels M, Joswig M et al. Peripheral blood mononuclear cells isolated from patients with diabetic nephropathy show increased activation of the oxidative-stress sensitive transcription factor NF-kappaB. Diabetologia. 1999; 42:222-32.

Ref ID: 9062
(232) Lee H A, Hughes D A. Alpha-lipoic acid modulates NF-kappaB activity in human monocytic cells by direct interaction with DNA. Exp Gerontol. 2002; 37:401-10.

Ref ID: 105768
(233) Zhang W J, Frei B. Alpha-lipoic acid inhibits TNF-alpha-induced NF-kappaB activation and adhesion molecule expression in human aortic endothelial cells. FASEB J. 2001; 15:2423-32.

Ref ID: 103806
(234) Fraunberger P, Grone E, Grone H J, Walli A K. Simvastatin reduces endotoxin-induced NF-kappaB activation and mortality in guinea pigs despite lowering circulating LDL cholesterol*. Shock. 2008.

Ref ID: 108791
(235) Guijarro C, Kim Y, Schoonover C M, Massy Z A, O'Donnell M P, Kasiske B L et al. Lovastatin inhibits lipopolysaccharide-induced NF-kappaB activation in human mesangial cells. Nephrol Dial Transplant. 1996; 11:990-996.

Ref ID: 108794
(236) Holschermann H, Schuster D, Parviz B, Haberbosch W, Tillmanns H, Muth H. Statins prevent NF-kappaB transactivation independently of the IKK-pathway in human endothelial cells. Atherosclerosis. 2006; 185:240-245.

Ref ID: 108792
(237) Ortego M, Bustos C, Hernandez-Presa M A, Tunon J, Diaz C, Hernandez G et al. Atorvastatin reduces NF-kappaB activation and chemokine expression in vascular smooth muscle cells and mononuclear cells. Atherosclerosis. 1999; 147:253-61.

Ref ID: 108793
(238) Ortego M, Gomez-Hernandez A, Vidal C, Sanchez-Galan E, Blanco-Colio L M, Martin-Ventura J L et al. HMG-CoA reductase inhibitors reduce I kappa B kinase activity induced by oxidative stress in monocytes and vascular smooth muscle cells. J Cardiovasc Pharmacol. 2005; 45:468-75.

Ref ID: 107582
(239) Griffith O W, Meister A. Origin and turnover of mitochondrial glutathione. Proc Natl Acad Sci USA. 1985; 82:4668-72.

Ref ID: 108737
(240) Hagen T M, Wierzbicka G T, Sillau A H, Bowman B B, Jones D P. Bioavailability of dietary glutathione: effect on plasma concentration. Am J Physiol. 1990; 259:524-9.

Ref ID: 13662
(241) Hagen T M, Ingersoll R T, Lykkesfeldt J, Liu J, Wehr C M, Vinarsky V et al. (R)-alpha-lipoic acid-supplemented old rats have improved mitochondrial function, decreased oxidative damage, and increased metabolic rate. FASEB J. 1999; 13:411-18.

Ref ID: 28182
(242) Powell L A, Warpeha K M, Xu W, Walker B, Trimble E R. High glucose decreases intracellular glutathione concentrations and upregulates inducible nitric oxide synthase gene expression in intestinal epithelial cells. J Mol Endocrinol. 2004; 33:797-803.

Ref ID: 108721
(243) Martina V, Bruno G A, Zumpano E, Origlia C, Quaranta L, Pescarmona G P. Administration of glutathione in patients with type 2 diabetes mellitus increases the platelet constitutive nitric oxide synthase activity and reduces PAI-1. J Endocrinol Invest. 2001; 24:37-41.

Ref ID: 108723
(244) Byun C H, Koh J M, Kim D K, Park S I, Lee K U, Kim G S. Alpha-lipoic acid inhibits TNF-alpha-induced apoptosis in human bone marrow stromal cells. J Bone Miner Res. 2005; 20:1125-35.

Ref ID: 106614
(245) Lee C K, Lee E Y, Kim Y G, Mun S H, Moon H B, Yoo B. Alpha-lipoic acid inhibits TNF-alpha induced NF-kappa B activation through blocking of MEKK1-MKK4-IKK signaling cascades. Int Immunopharmacol. 2008; 8:362-70.

Ref ID: 108736
(246) Jiang J L, Jiang D J, Tang Y H, Li N S, Deng H W, Li Y J. Effect of simvastatin on endothelium-dependent vaso-relaxation and endogenous nitric oxide synthase inhibitor. Acta Pharmacol Sin. 2004; 25:893-901.

Ref ID: 108646
(247) Camera A, Hopps E, Caimi G. Diabetic microangiopathy: physiopathological, clinical and therapeutic aspects. Minerva Endocrinol. 2007; 32:209-29.

Ref ID: 108743
(248) Cameron N, Cotter M, Inkster M, Nangle M. Looking to the future: diabetic neuropathy and effects of rosuvastatin on neurovascular function in diabetes models. Diabetes Res Clin Pract. 2003; 61 Suppl 1:S35-S39.

Ref ID: 108746
(249) Fried L F, Forrest K Y, Ellis D, Chang Y, Silvers N, Orchard T J. Lipid modulation in insulin-dependent diabetes mellitus: effect on microvascular outcomes. J Diabetes Complications. 2001; 15:113-19.

Ref ID: 108742
(250) Haslbeck M. [Diabetic neuropathy: new therapeutic options?]. MMW Fortschr Med. 2006; 148:41-44.

Ref ID: 108744
(251) Ii M, Nishimura H, Kusano K F, Qin G, Yoon Y S, Wecker A et al. Neuronal nitric oxide synthase mediates statin-induced restoration of vasa nervorum and reversal of diabetic neuropathy. Circulation. 2005; 112:93-102.

Ref ID: 108745
(252) Lacigova S, Rusavy Z, Cechurova D, Jankovec Z, Zourek M. [Autonomic neuropathy in diabetics, treatment possibilities]. Vnitr Lek. 2002; 48:534-41.

Ref ID: 108718
(253) Keegan A, Cotter M A, Cameron N E. Corpus cavernosum dysfunction in diabetic rats: effects of combined alpha-lipoic acid and gamma-linolenic acid treatment. Diabetes Metab Res Rev. 2001; 17:380-386.

Ref ID: 107583
(254) Bush P A, Gonzalez N E, Ignarro L J. Biosynthesis of nitric oxide and citrulline from L-arginine by constitutive nitric oxide synthase present in rabbit corpus cavernosum. Biochem Biophys Res Commun. 1992; 186:308-14.

Ref ID: 108747
(255) Sener G, Paskaloglu K, Satiroglu H, Alican I, Kacmaz A, Sakarcan A. L-carnitine ameliorates oxidative damage due to chronic renal failure in rats. J Cardiovasc Pharmacol. 2004; 43 :698-705.

Ref ID: 108748
(256) Da R R, Assaloni R, Ceriello A. Molecular targets of diabetic vascular complications and potential new drugs. Curr Drug Targets. 2005; 6:503-9.

Ref ID: 107581
(257) Zou P, Hou P, Oh S S, Ge X, Bloodworth B C, Low M Y et al. Identification of benzamidenafil, a new class of phosphodiesterase-5 inhibitor, as an adulterant in a dietary supplement. J Pharm Biomed Anal. 2008; 47:255-59.

Ref ID: 108871

What is claimed:

1. A unit dosage form as an adjunct to HMG-CoA reductase inhibitor (statin) therapy by supporting mitochondrial metabolism as a method for improving the effectiveness and ameliorating the side effects of statins, said unit dosage form consisting of a single-layer tablet comprising as an active ingredient a member selected from the group consisting of:
L-arginine, in a therapeutically effective amount ranging from about 75 mg/day to about 4000 mg/day,
tetrahydrobiopterin, in a therapeutically effective amount ranging from about 24 mg/day to about 3000 mg/day,
Coenzyme Q10, in a therapeutically effective amount ranging from about 5 mg/day to about 1200 mg/day,
D-alpha-lipoic acid, in a therapeutically effective amount ranging from about 30 mg/day to about 1500 mg/day,
acetyl-L-carnitine, in a therapeutically effective amount ranging from about 90 mg/day to about 4000 mg/day,
and combinations thereof.

2. The unit dosage foam of claim 1 wherein said active ingredient is a combination of L-arginine in a therapeutically effective amount ranging from about 75 mg/day to about 4000 mg/day and tetrahydrobiopterin in a therapeutically effective amount ranging from about 24 mg/day to about 3000 mg/day.

3. The unit dosage form of claim 1 wherein said active ingredient is a combination of tetrahydrobiopterin in a therapeutically effective amount ranging from about 24 mg/day to about 3000 mg/day and coenzyme Q10 in a therapeutically effective amount ranging from about 5 mg/day to about 1200 mg/day.

4. The unit dosage form of claim 1 wherein said active ingredient is a combination of tetrahydrobiopterin in a therapeutically effective amount ranging from about 24 mg/day to about 3000 mg/day and D-alpha-lipoic acid in a therapeutically effective amount ranging from about 30 mg/day to about 1500 mg.

5. The unit dosage form of claim 1 wherein said active ingredient is a combination of tetrahydrobiopterin in a therapeutically effective amount ranging from about 24 mg/day to about 3000 mg/day and acetyl-L-carnitine in a therapeutically effective amount ranging from about 15 mg/day to about 300 mg/day.

6. The unit dosage form of claim 1 wherein said active ingredient is a combination of Coenzyme Q in a therapeutically effective amount ranging from about 5 mg/day to about 1200 mg/day and L-arginine in a therapeutically effective amount ranging from about 90 mg/day to about 4000 mg/day.

7. The unit dosage form of claim 1 wherein said active ingredient is a combination of D-alpha-lipoic acid in a therapeutically effective amount ranging from about 30 mg/day to about 1500 mg/day and L-arginine in a therapeutically effective amount ranging from about 75 mg/day to about 4000 mg/day.

8. The unit dosage form of claim 1 wherein said active ingredient is a combination of L-arginine in a therapeutically effective amount ranging from about 90 mg/day to about 4000 mg/day and acetyl-L-carnitine in a therapeutically effective amount ranging from about 90 mg/day to about 4000 mg/day.

9. The unit dosage form of claim 1 wherein said active ingredient is a combination of Coenzyme Q in a therapeutically effective amount ranging from about 5 mg/day to about 1200 mg/day and D-alpha-lipoic acid in a therapeutically effective amount ranging from about 30 mg/day to about 1500 mg/day.

10. The unit dosage form of claim 1 wherein said active ingredient is a combination of Coenzyme Q in a therapeutically effective amount ranging from about 5 mg/day to about 1200 mg/day and acetyl-L-carnitine in a therapeutically effective amount ranging from about 90 mg/day to about 4000 mg/day.

11. The unit dosage form of claim 1 wherein said active ingredient is a combination of acetyl-L-carnitine, in a therapeutically effective amount ranging from about 90 mg/day to about 4000 mg/day and D-alpha-lipoic acid in a therapeutically effective amount ranging from about 30 mg/day to about 1500 mg/day.

12. The unit dosage form of claim 1 wherein said active ingredient is a combination of tetrahydrobiopterin in a therapeutically effective amount ranging from about 24 mg/day to about 3000 mg/day, coenzyme Q10 in a therapeutically effective amount ranging from about 5 mg/day to about 1200 mg/day, and L-arginine in a therapeutically effective amount ranging from about 75 mg/day to about 4000 mg/day.

13. The unit dosage form of claim 1 wherein said active ingredient is a combination of tetrahydrobiopterin in a therapeutically effective amount ranging from about 24 mg/day to about 3000 mg/day, coenzyme Q10 in a therapeutically effective amount ranging from about 5 mg/day to about 1200 mg/day, and D-alpha-lipoic acid in a therapeutically effective amount ranging from about 30 mg/day to about 1500 mg/day.

14. The unit dosage form of claim 1 wherein said active ingredient is a combination of tetrahydrobiopterin in a therapeutically effective amount ranging from about 24 mg/day to about 3000 mg/day, coenzyme Q10 in a therapeutically effective amount ranging from about 5 mg/day to about 1200 mg/day, and acetyl-L-carnitine in a therapeutically effective amount ranging from about 15 mg/day to about 300 mg/day.

15. The unit dosage form of claim 1 wherein said active ingredient is a combination of Coenzyme Q in a therapeutically effective amount ranging from about 5 mg/day to about 1200 mg/day, L-arginine in a therapeutically effective amount ranging from about 90 mg/day to about 4000 mg/day, and D-alpha-lipoic acid in a therapeutically effective amount ranging from about 90 mg/day to about 4000 mg.

16. The unit dosage form of claim 1 wherein said active ingredient is a combination of Coenzyme Q in a therapeutically effective amount ranging from about 5 mg/day to about 1200 mg/day, D-alpha-lipoic acid in a therapeutically effective amount ranging from about 30 mg/day to about 1500 mg/day, and acetyl-L-carnitine in a therapeutically effective amount ranging from about 90 mg/day to about 4000 mg/day.

17. The unit dosage form of claim 1 wherein said active ingredient is a combination of Coenzyme Q in a therapeutically effective amount ranging from about 5 mg/day to about 1200 mg/day, acetyl-L-carnitine in a therapeutically effective amount ranging from about 90 mg/day to about 4000 mg/day, and L-arginine in a therapeutically effective amount ranging from about 75 mg/day to about 4000 mg/day.

18. The unit dosage form of claim 1 wherein said active ingredient is a combination ofL-arginine in a therapeutically effective amount ranging from about 75 mg/day to about 4000 mg/day, and tetrahydrobiopterin in a therapeutically effective amount ranging from about 24 mg/day to about 3000 mg/day, and D-alpha-lipoic acid in a therapeutically effective amount ranging from about 30 mg/day to about 1500 mg.

19. The unit dosage form of claim 1 wherein said active ingredient is a combination oftetrahydrobiopterin in a therapeutically effective amount ranging from about 24 mg/day to about 3000 mg/day, D-alpha-lipoic acid in a therapeutically effective amount ranging from about 30 mg/day to about 1500 mg, and acetyl-L-carnitine in a therapeutically effective amount ranging from about 90 mg/day to about 4000 mg/day.

20. The unit dosage form of claim 1 wherein said active ingredient is a combination of tetrahydrobiopterin in a therapeutically effective amount ranging from about 24 mg/day to about 3000 mg/day, acetyl-L-carnitine in a therapeutically effective amount ranging from about 15 mg/day to about 300 mg/day, and L-arginine in a therapeutically effective amount ranging from about 75 mg/day to about 4000 mg/day.

21. The unit dosage form of claim 1 wherein said active ingredient is a combination of acetyl-L-carnitine, in a therapeutically effective amount ranging from about 90 mg/day to about 4000 mg/day, D-alpha-lipoic acid in a therapeutically effective amount ranging from about 30 mg/day to about 1500 mg/day, and L-arginine in a therapeutically effective amount ranging from about 75 mg/day to about 4000 mg/day.

22. The unit dosage form of claim 1 wherein said active ingredient is a combination of tetrahydrobiopterin in a therapeutically effective amount ranging from about 24 mg/day to about 3000 mg/day, coenzyme Q10 in a therapeutically effective amount ranging from about 5 mg/day to about 1200 mg/day, and D-alpha-lipoic acid in a therapeutically effective amount ranging from about 30 mg/day to about 1500 mg/day.

23. The unit dosage faun of claim 1 wherein said active ingredient is a combination of tetrahydrobiopterin in a therapeutically effective amount ranging from about 24 mg/day to about 3000 mg/day, coenzyme Q10 in a therapeutically effective amount ranging from about 5 mg/day to about 1200 mg/day, D-alpha-lipoic acid in a therapeutically effective amount ranging from about 30 mg/day to about 1500 mg/day, and acetyl-L-carnitine in a therapeutically effective amount ranging from about 90 mg/day to about 4000 mg/day.

24. The unit dosage form of claim 1 wherein said active ingredient is a combination of tetrahydrobiopterin in a therapeutically effective amount ranging from about 24 mg/day to about 3000 mg/day, coenzyme Q10 in a therapeutically effective amount ranging from about 5 mg/day to about 1200 mg/day, acetyl-L-camitine in a therapeutically effective amount ranging from about 15 mg/day to about 300 mg/day, and L-arginine in a therapeutically effective amount ranging from about 75 mg/day to about 4000 mg/day.

25. The unit dosage form of claim 1 wherein said active ingredient is a combination of tetrahydrobiopterin in a therapeutically effective amount ranging from about 24 mg/day to about 3000 mg/day, coenzyme Q10 in a therapeutically effective amount ranging from about 5 mg/day to about 1200 mg/day, acetyl-L-carnitine in a therapeutically effective amount ranging from about 15 mg/day to about 300 mg/day, L-arginine in a therapeutically effective amount ranging from about 75 mg/day to about 4000 mg/day, and D-alpha-lipoic acid in an amount ranging from about 30 mg/day to about 1500 mg/day.

26. A unit dosage form containing an HMG-CoA reductase inhibitor (statin), and adjuncts which support mitochondrial metabolism, in therapeutically effective amounts for the treatment of conditions requiring statin therapy, said unit dosage form consisting of a single-layer tablet comprising as active ingredients (a) atorvastatin in an amount ranging from about 5 mg/day to about 160 mg/day and (b) a member selected from the group consisting of
tetrahydrobiopterin in an amount ranging from about 24 mg/day to about 3000 mg/day,
Coenzyme Q10 in an amount ranging from about 5 mg/day to about 1200 mg/day,
D-alpha-lipoic acid in an amount ranging from about 30 mg/day to about 1500 mg/day,
L-arginine in an amount ranging from about 75 mg/day to about 4000 mg/day,
acetyl-L-carnitine in an amount ranging from about 90 mg/day to about 4000 mg/day,
and combinations thereof.

27. The unit dosage foiiii of claim 26 wherein active ingredient (b) is a combination of tetrahydrobiopterin in an amount ranging from about 24 mg/day to about 3000 mg/day and Coenzyme Q10 in an amount ranging from about 5 mg/day to about 1200 mg/day.

28. The unit dosage form of claim 26 wherein active ingredient (b) is a combination of tetrahydrobiopterin in an amount ranging from about 24 mg/day to about 3000 mg/day and D-alpha-lipoic acid in an amount ranging from about 30 mg/day to about 1500 mg/day.

29. The unit dosage form of claim 26 wherein active ingredient (b) is a combination of tetrahydrobiopterin in an amount ranging from about 24 mg/day to about 3000 mg/day and L-arginine in an amount ranging from about 75 mg/day to about 4000 mg/day.

30. The unit dosage form of claim 26 wherein active ingredient (b) is a combination of tetrahydrobiopterin in an amount ranging from about 24 mg/day to about 3000 mg/day and acetyl-L-carnitine in an amount ranging from about 90 mg/day to about 4000 mg/day.

31. The unit dosage form of claim 26 wherein active ingredient (b) is a combination of Coenzyme Q10 in an amount ranging from about 5 mg/day to about 1200 mg/day and D-alpha-lipoic acid in an amount ranging from about 30 mg/day to about 1500 mg/day.

32. The unit dosage form of claim 26 wherein active ingredient (b) is a combination of Coenzyme Q10 in an amount ranging from about 5 mg/day to about 1200 mg/day and L-arginine in an amount ranging from about 75 mg/day to about 4000 mg/day.

33. The unit dosage foiiii of claim 26 wherein active ingredient (b) is a combination of Coenzyme Q10 in an amount ranging from about 5 mg/day to about 1200 mg/day and acetyl-L-carnitine in an amount ranging from about 90 mg/day to about 4000 mg/day.

34. The unit dosage form of claim 26 wherein active ingredient (b) is a combination of D-alpha-lipoic acid in an amount ranging from about 30 mg/day to about 1500 mg/day and L-arginine in an amount ranging from about 75 mg/day to about 4000 mg/day.

35. The unit dosage form of claim 26 wherein active ingredient (b) is a combination of D-alpha-lipoic acid in an amount ranging from about 30 mg/day to about 1500 mg/day and L-arginine in an amount ranging from about 75 mg/day to about 4000 mg/day and acetyl-L-carnitine in an amount ranging from about 90 mg/day to about 4000 mg/day.

36. The unit dosage form of claim 26 wherein active ingredient (b) is a combination of L-arginine in an amount ranging from about 75 mg/day to about 4000 mg/day and acetyl-L-carnitine in an amount ranging from about 90 mg/day to about 4000 mg/day.

37. The unit dosage form of claim 26 wherein active ingredient (b) is a combination of tetrahydrobiopterin in an amount ranging from about 24 mg/day to about 3000 mg/day, Coenzyme Q10 in an amount ranging from about 5 mg/day to about 1200 mg/day, and D-alpha-lipoic acid in an amount ranging from about 30 mg/day to about 1500 mg/day.

38. The unit dosage form of claim 26 wherein active ingredient (b) is a combination of tetrahydrobiopterin in an amount ranging from about 24 mg/day to about 3000 mg/day, Coenzyme Q10 in an amount ranging from about 5 mg/day to about 1200 mg/day, and L-arginine in an amount ranging from about 75 mg/day to about 4000 mg/day.

39. The unit dosage form of claim 26 wherein active ingredient (b) is a combination of tetrahydrobiopterin in an amount ranging from about 24 mg/day to about 3000 mg/day, Coenzyme Q10 in an amount ranging from about 5 mg/day to about 1200 mg/day, and acetyl-L-carnitine in an amount ranging from about 90 mg/day to about 4000 mg/day.

40. The unit dosage form of claim 26 wherein active ingredient (b) is a combination of tetrahydrobiopterin in an amount ranging from about 24 mg/day to about 3000 mg/day, D-alpha-lipoic acid in an amount ranging from about 30 mg/day to about 1500 mg/day, and L-arginine in an amount ranging from about 75 mg/day to about 4000 mg/day.

41. The unit dosage form of claim 26 wherein active ingredient (b) is a combination of tetrahydrobiopterin in an amount ranging from about 24 mg/day to about 3000 mg/day, D-alpha-lipoic acid in an amount ranging from about 30 mg/day to about 1500 mg/day, and acetyl-L-carnitine in an amount ranging from about 90 mg/day to about 4000 mg/day.

42. The unit dosage form of claim 26 wherein active ingredient (b) is a combination of tetrahydrobiopterin in an amount ranging from about 24 mg/day to about 3000 mg/day, D-alpha-lipoic acid in an amount ranging from about 30 mg/day to about 1500 mg/day, L-arginine in an amount ranging from about 75 mg/day to about 4000 mg/day, and acetyl-L-carnitine in an amount ranging from about 90 mg/day to about 4000 mg/day.

43. The unit dosage form of claim 26 wherein active ingredient (b) is a combination of tetrahydrobiopterin in an amount ranging from about 24 mg/day to about 3000 mg/day, Coenzyme Q10 in an amount ranging from about 5 mg/day to about 1200 mg/day, D-alpha-lipoic acid in an amount ranging from about 30 mg/day to about 1500 mg/day, and L-arginine in an amount ranging from about 75 mg/day to about 4000 mg/day.

44. The unit dosage form of claim 26 wherein active ingredient (b) is a combination of tetrahydrobiopterin in an amount ranging from about 24 mg/day to about 3000 mg/day, Coenzyme Q10 in an amount ranging from about 5 mg/day to about 1200 mg/day, D-alpha-lipoic acid in an amount ranging from about 30 mg/day to about 1500 mg/day, and acetyl-L-carnitine in an amount ranging from about 90 mg/day to about 4000 mg/day.

45. The unit dosage form of claim 26 wherein active ingredient (b) is a combination of tetrahydrobiopterin in an amount ranging from about 24 mg/day to about 3000 mg/day, Coenzyme Q10 in an amount ranging from about 5 mg/day to about 1200 mg/day, L-arginine in an amount ranging from about 75 mg/day to about 4000 mg/day, and acetyl-L-carnitine in an amount ranging from about 15 mg/day to about 300 mg/day.

46. The unit dosage form of claim 26 wherein active ingredient (b) is a combination of tetrahydrobiopterin in an amount ranging from about 24 mg/day to about 3000 mg/day, Coenzyme Q10 in an amount ranging from about 5 mg/day to about 1200 mg/day, D-alpha-lipoic acid in an amount ranging from about 30 mg/day to about 1500 mg/day, L-arginine in an amount ranging from about 75 mg/day to about 4000 mg/day, and acetyl-L-carnitine in an amount ranging from about 90 mg/day to about 4000 mg/day.

47. The unit dosage form of claim 26 wherein said atorvastatin is a member selected from the group consisting of atorvastatin, pravastatin, rosuvastatin, pitavastatin, simvastatin, fluvastatin and lovastatin.

48. A method for treating a patient using adjunctive active ingredients to increase the clinical efficiency of a selective phosphodiesterase 5 inhibitor currently being used by said patient for the management and clinical amelioration of erectile dysfunction, said method comprising administering to said patient a single-layer tablet with a therapeutically effective amount of an active ingredient selected from the group consisting of
Coenzyme Q10 in an amount ranging from about 5 mg/day to about 1200 mg/day,
D-alpha-lipoic acid in an amount ranging from about 30 mg/day to about 1500 mg/day, and
tetrahydrobiopterin in an amount ranging from about 24 mg/day to about 3000 mg/day.

49. The method of claim 48 further comprising as an active ingredient L-arginine in an amount ranging from about 75 mg/day to about 4000 mg/day.

50. The method of claim 48 wherein active ingredient (b) is tetrahydrobiopterin in an amount ranging from about 24 mg/day to about 3000 mg/day.

51. The method of claim 48 wherein active ingredient (b) is D-alpha-lipoic acid in an amount ranging from about 30 mg/day to about 1500 mg/day.

52. The method of claim 48 wherein active ingredient (b) is tetrhydrobiopterin in an amount ranging from about 24 mg/day to about 3000 mg/day and further comprising as an active ingredient L-arginine in an amount ranging from about 75 mg/day to about 4000 mg/day.

53. The method of claim 48 wherein active ingredient (b) is D-alpha-lipoic acid in an amount ranging from about 30 mg/day to about 1500 mg/day and further comprising as an active ingredient L-arginine in an amount ranging from about 75 mg/day to about 4000 mg/day.

54. A method for treating a patient using adjunctive active ingredients simultaneously with a selective phosphodiesterase 5 inhibitor useful for the clinical amelioration of erectile dysfunction, said method comprising administering to said patient a single-layer tablet containing active ingredients comprising (a) a therapeutically effective amount of sildenafil in an amount ranging from 0.5 mg/day to about 300 mg/day and (b) a therapeutically effective amount of a member selected from the group consisting of
Coenzyme Q10 in an amount ranging from about 5 mg/day to about 1200mg/day,
tetrahydrobiopterin in an amount ranging from about 24 mg/day to about 3000 mg/day, and
D-alpha-lipoic acid in an amount ranging from about 30 mg/day to about 1500 mg/day.

55. The method of claim 54 wherein active ingredient (b) is Coenzyme Q10 in an amount ranging from about 5 mg/day to about 1200mg/day and said active ingredients further comprise a selective L-arginine in an amount ranging from about 75 mg/day to about 4000 mg/day.

56. The method of claim 54 wherein active ingredient (b) is a combination of Coenzyme Q10 in an amount ranging from about 5 mg/day to about 1200mg/day and tetrahydrobiopterin in an amount ranging from about 24 mg/day to about 3000 mg/day.

57. The method of claim 54 wherein active ingredient (b) is a combination of Coenzyme Q10 in an amount ranging from about 5 mg/day to about 1200mg/day and D-alpha-lipoic acid in an amount ranging from about 30 mg/day to about 1500 mg/day.

58. The method of claim 54 wherein active ingredient (b) is tetrahydrobiopterin in an amount ranging from about 24 mg/day to about 3000 mg/day, and said active ingredients further comprise L-arginine in an amount ranging from about 75 mg/day to about 4000 mg/day.

59. The method of claim 54 wherein active ingredient (b) is D-alpha-lipoic acid in an amount ranging from about 30 mg/day to about 1500 mg/day and further comprising as an active ingredient L-arginine in an amount ranging from about 75 mg/day to about 4000 mg/day.

60. The method of claim 54 wherein active ingredient (b) is Coenzyme Q10 in an amount ranging from about 5 mg/day to about 1200mg/day and said active ingredients further comprise D-alpha-lipoic acid in an amount ranging from about 30 mg/day to about 1500 mg/day.

61. The method of claim 54 wherein active ingredient (b) is a combination of Coenzyme Q10 in an amount ranging from about 5 mg/day to about 1200mg/day and tetrahydrobiopterin in an amount ranging from about 24 mg/day to about 3000 mg/day and said active ingredients further comprise a selective L-arginine in an amount ranging from about 75 mg/day to about 4000 mg/day.

62. The method of claim 54 wherein active ingredient (b) is a combination of Coenzyme Q10 in an amount ranging from about 5 mg/day to about 1200mg/day and tetrahydrobiopterin in an amount ranging from about 24 mg/day to about 3000 mg/day, and D-alpha-lipoic acid in an amount ranging from about 30 mg/day to about 1500 mg/day.

63. The method of claim 54 wherein active ingredient (b) is a combination of tetrahydrobiopterin in an amount ranging from about 24 mg/day to about 3000 mg/day and tetrahydrobiopterin in an amount ranging from about 24 mg/day to about 3000 mg/day, and D-alpha-lipoic acid in an amount ranging from about 30 mg/day to about 1500 mg/day.

64. A method for treating a patient using a selective phosphodiesterase 5 inhibitor useful for the clinical amelioration of erectile dysfunction and an HMG-CoA reductase inhibitor (statin), said method comprising simultaneously administering to said patient a single-layer tablet comprising a therapeutically effective amount of sildenafil in an amount ranging from 0.5 mg/day to about 300 mg/day and a therapeutically effective amount of atorvastatin in an amount ranging from about 5 mg/day to about 160 mg/day.

65. The method of claim 64 wherein said single layer tablet further comprises as an active ingredient L-arginine in an amount ranging from about 75 mg/day to about 4000 mg/day.

66. The method of claim 64 wherein said single layer tablet further comprises as an active ingredient Coenzyme Q10 in an amount ranging from about 5 mg/day to about 1200 mg/day.

67. The method of claim 64 wherein said single layer tablet further comprises as active ingredients Coenzyme Q10 in an amount ranging from about 5 mg/day to about 1200 mg/day and D-alpha-lipoic acid in an amount ranging from about 30 mg/day to about 1500 mg/day.

68. The method of claim 64 wherein said single layer tablet further comprises as an active ingredient tetrahydrobiopterin in an amount ranging from about 24 mg/day to about 3000 mg/day.

69. The method of claim 64 wherein said single layer tablet further comprises as active ingredients L-arginine in an amount ranging from about 75 mg/day to about 4000 mg/day, Coenzyme Q10 in an amount ranging from about 5 mg/day to about 1200 mg/day, and D-alpha-lipoic acid in an amount ranging from about 30 mg/day to about 1500 mg/day.

70. The method of claim 64 wherein said single layer tablet further comprises as active ingredients L-arginine in an amount ranging from about 75 mg/day to about 4000 mg/day and tetrahydrobiopterin in an amount ranging from about 24 mg/day to about 3000 mg/day.

71. The method of claim 64 wherein said single layer tablet further comprises as active ingredients L-arginine in an amount ranging from about 75 mg/day to about 4000 mg/day and D-alpha-lipoic acid in an amount ranging from about 30 mg/day to about 1500 mg/day.

72. The method of claim 64 wherein said single layer tablet further comprises as active ingredients Coenzyme Q10 in an amount ranging from about 5 mg/day to about 1200 mg/day and tetrahydrobiopterin in an amount ranging from about 24 mg/day to about 3000 mg/day.

73. The method of claim 64 wherein said single layer tablet further comprises as active ingredients Coenzyme Q10 in an amount ranging from about 5 mg/day to about 1200 mg/day and D-alpha-lipoic acid in an amount ranging from about 30 mg/day to about 1500 mg/day.

74. The method of claim 64 wherein said single layer tablet further comprises as active ingredients Coenzyme Q10 in an amount ranging from about 5 mg/day to about 1200 mg/day, D-alpha-lipoic acid in an amount ranging from about 30 mg/day to about 1500 mg/day, and tetrahydrobiopterin in an amount ranging from about 24 mg/day to about 3000 mg/day.

75. The method of claim 64 wherein said single layer tablet further comprises as active ingredients L-arginine in an amount ranging from about 75 mg/day to about 4000 mg/day, D-alpha-lipoic acid in an amount ranging from about 30 mg/day to about 1500 mg/day, and tetrahydrobiopterin in an amount ranging from about 24 mg/day to about 3000 mg/day.

76. The method of claim 64 or 74 wherein said sildenafil is in the form of a member selected from the group consisting of sildenafil, tadalafil, vardenafil, udenafil, avanafil and benzamidenafil.

77. The unit dosage form of claim 1 or 26 wherein said L-arginine is in the form of a member selected from the group consisting of L-arginine, L-arginine ascorbate, L-arginine citrate or L-arginine and an anion selected from the group consisting of hydroxide, halide or acetate, and derivatives and salts thereof.

78. The method of claim 48, 54, or 64 wherein said L-arginine is in the form of a member selected from the group consisting of L-arginine, L-arginine ascorbate, L-arginine citrate or L-arginine and an anion selected from the group consisting of hydroxide, halide or acetate, and derivatives and salts thereof.

79. The unit dosage form of claim 1 or 26 wherein said tetrahydrobiopterin is in the form of a member selected from the group consisting of 6-lactyl-7′,8′-dihydropterin (sepiapterin), 6-1′,2′-dioxypropyl tetrahydropterin(6-pyruvoyltetrahydropterin), 6-1′-oxo-2′-hydroxypropyl tetrahydropterin(6-lactoyltetrahydropterin), and 6-1′-hydroxy-2′-oxypropyl tetrahydropterin(6-hydroxypropy)tetrahydropterin), (6R)-L-erythro-5,6,7,8-tetrahydrobiopterin (BH4), (6R,S)-5,6,7,8-tetrahydrobiopterin, 1′,2′-diacetyl-5,6,7,8-tetrahydrobiopterin sepiapterin, 6-methyl-5,6,7,8-tetrahydropterin 6-hydroxymethyl-5,6,7,8-tetrahydropterin, 6-phenyl-5,6,7,8-tetrahydropterin, and precursors thereof.

80. The method of claim 48 or 54 wherein said tetrahydrobiopterin is in the form of a member selected from the group consisting of 6-lactyl-7′,8′-dihydropterin (sepiapterin), 6-1′,2′-dioxypropyl tetrahydropterin(6-pyruvoyltetrahydropterin), 6-1′-oxo-2′-hydroxypropyl tetrahydropterin(6-lactoyltetrahydropterin), and 6-1′-hydroxy-2′-oxypropyl tetrahydropterin(6-hydroxypropy)tetrahydropterin), (6R)-L-erythro-5,6,7,8-tetrahydrobiopterin (BH4), (6R,S)-5,6,7,8-tetrahydrobiopterin, 1′,2′-diacetyl-5,6,7,8-tetrahydrobiopterin sepiapterin, 6-methyl-5,6,7,8-tetrahydropterin 6-hydroxymethyl-5,6,7,8-tetrahydropterin, 6-phenyl-5,6,7,8-tetrahydropterin, and precursors thereof.

81. The method of claim 64 wherein said single layer tablet further comprises as an active ingredient tetrahydrobiopterin in the form of a member selected from the group consisting of 6-lactyl-7′,8′-dihydropterin (sepiapterin), 6-1′,2′-dioxypropyl tetrahydropterin (6-pyruvoyltetrahydropterin), 6-1′-oxo-2′-hydroxypropyl tetrahydropterin(6-lactoyltetrahydropterin), and 6-1′-hydroxy-2′-oxypropyl tetrahydropterin(6-hydroxypropy)tetrahydropterin), (6R)-L-erythro-5,6,7,8-tetrahydrobiopterin (BH4), (6R,S)-5,6,7,8-tetrahydrobiopterin, 1′,2′-diacetyl-5,6,7,8-tetrahydrobiopterin sepiapterin, 6-methyl-5,6,7,8-tetrahydropterin 6-hydroxymethyl-5,6,7,8-tetrahydropterin, 6-phenyl-5,6,7,8-tetrahydropterin, and precursors thereof.

82. The unit dosage of claim 1 or 26 wherein said D-alpha-lipoic acid is in the form of a member selected from the group consisting of D-alpha-lipoic acid, dihydrolipoic acid, and derivatives and salts thereof.

83. The method of claim 48 or 54 wherein said D-alpha-lipoic acid is in the form of a member selected from the group consisting of D-alpha-lipoic acid, dihydrolipoic acid, and derivatives and salts thereof.

84. The method of claim 64 wherein said single layer tablet further comprises as an active ingredient D-alpha-lipoic acid in the form of a member selected from the group consisting of D-alpha-lipoic acid, dihydrolipoic acid, and derivatives and salts thereof.

85. The unit dosage of claim 1 or 26 wherein said acetyl-L-carnitine is in the form of a member selected from the group consisting of Acetyl-L-carnitine, propionyl-L-carnitine, L-carnitine ascorbate, L-carnitine citrate or L-carnitine and an anion selected from the. group consisting of hydroxide, halide or acetate, and derivatives and salts thereof.

86. The method of claim 64 wherein said atorvastatin is in the form of a member selected from the group consisting of atorvastatin, pravastatin, rosuvastatin, pitavastatin, simvastatin, fluvastatin and lovastatin.
